SEQUENCE LISTING (114 sequences)

SEQ ID NO: 1 - protein, 527 aa, Homo sapiens
MESAAALHFSRPASLLLLLLSLCALVSAQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVSPCAVALPIIVVILMIPIAVCIYWINKLQKEKKILSGEKEFERETREIALKELEKERVQKEEELQVKEKLQEELRWRRTFLHAVDVVLDPDTAHPDLFLSEDRRSVRRCPFRHLGESVPDNPERFDSQPCVLGRESFASGKHYWEVEVENVIEWTVGVCRDSVERKGEVLLIPQNGFWTLEMHKGQYRAVSSPDRILPLKESLCRVGVFLDYEAGDVSFYNMRDRSHIYTCPRSAFSVPVRPFFRLGCEDSPIFICPALTGANGVTVPEEGLTLHRVGTHQSL

SEQ ID NO: 2 - protein, 523 aa, Homo sapiens
MEPAAALHFSLPASLLLLLLLLLLSLCALVSAQFTVVGPANPILAMVGENTTLRCHLSPEKNAEDMEVRWFRSQFSPAVFVYKGGRERTEEQMEEYRGRITFVSKDINRGSVALVIHNVTAQENGIYRCYFQEGRSYDEAILRLVVAGLGSKPLIEIKAQEDGSIWLECISGGWYPEPLTVWRDPYGEVVPALKEVSIADADGLFMVTTAVIIRDKYVRNVSCSVNNTLLGQEKETVIFIPESFMPSASPWMVALAVILTASPWMVSMTVILAVFIIFMAVSICCIKKLQREKKILSGEKKVEQEEKEIAQQLQEELRWRRTFLHAADVVLDPDTAHPELFLSEDRRSVRRGPYRQRVPDNPERFDSQPCVLGWESFASGKHYWEVEVENVMVWTVGVCRHSVERKGEVLLIPQNGFWTLEMFGNQYRALSSPERILPLKESLCRVGVFLDYEAGDVSFYNMRDRSHIYTCPRSAFTVPVRPFFRLGSDDSPIFICPALTGASGVMVPEEGLKLHRVGTHQSL

SEQ ID NO: 3 - protein, 5 aa, Homo sapiens
SYDIN

SEQ ID NO: 4 - protein, 17 aa, Homo sapiens
WIFPGDDSIIQNEKFKG

SEQ ID NO: 5 - protein, 9 aa, Homo sapiens
LGPLRGFTY

SEQ ID NO: 6 - protein, 15 aa, Homo sapiens
RASESVDRYGSSFMH

SEQ ID NO: 7 - protein, 7 aa, Homo sapiens
RASNLES

SEQ ID NO: 8 - protein, 9 aa, Homo sapiens
QQSNEDPWT

SEQ ID NO: 9 - protein, 137 aa, Homo sapiens
MGWSWVFLFLLSVTAGVHSQVQLQQSGAELVKPGASVKLSCKASGYIFTSYDINWVRQRPEQGLEWIGWIFPGDDSIIQNEKFKGKATLTTDKSSSTVYMQLSRLTSEDSAVYFCARLGPLRGFTYWGQGTLVTVSA

SEQ ID NO: 10 - protein, 131 aa, Homo sapiens
METDTLLLWVLLLWVPGSTGDIVLTQSPASLAVSLGQRATISCRASESVDRYGSSFMHWYQQKPGQPPKLLIYRASNLESGIPARFSGSGSRTDFTLTINPVEADDVATYYCQQSNEDPWTFGGGTKLEIK

SEQ ID NO: 11 - protein, 5 aa, Homo sapiens
DYSMN

SEQ ID NO: 12 - protein, 17 aa, Homo sapiens
RINTETGEPTYADDFKG

SEQ ID NO: 13 - protein, 5 aa, Homo sapiens
DYAKR

SEQ ID NO: 14 - protein, 11 aa, Homo sapiens
KASQDVITAVA

SEQ ID NO: 15 - protein, 7 aa, Homo sapiens
STSYRYT

SEQ ID NO: 16 - protein, 9 aa, Homo sapiens
LQHYTTPWT

SEQ ID NO: 17 - protein, 133 aa, Homo sapiens
MAWVWTLLFLMAAAQSIQAQIQLVQSGPELKKPGETVKISCKASGYTFTDYSMNWVKQAPGKGLKWVGRINTETGEPTYADDFKGRFAFSLETSASTAYLQIKNLKNEDTATYFCVRDYAKRWGQGTSVTVSS

SEQ ID NO: 18 - protein, 131 aa, Homo sapiens
MGIKMESQIQVFVFVSLWLSGVDGDIVMTQSHKFMSTSVGDRVSITCKASQDVITAVAWYQQKPGQSPKLLIYSTSYRYTGVPDRFTGSGSGTDFTFTISSVQAEDLAVYYCLQHYTTPWTFGGGTKLEIK

SEQ ID NO: 19 - protein, 5 aa, Homo sapiens
SYWIE

SEQ ID NO: 20 - protein, 17 aa, Homo sapiens
EILPGSGSTKYNEKFRG

SEQ ID NO: 21 - protein, 12 aa, Homo sapiens
LKGYYGGGAMDY

SEQ ID NO: 22 - protein, 11 aa, Homo sapiens
RASKSISKYLA

SEQ ID NO: 23 - protein, 7 aa, Homo sapiens
SGSTLQS

SEQ ID NO: 24 - protein, 9 aa, Homo sapiens
QQHNEYPWT

SEQ ID NO: 25 - protein, 140 aa, Homo sapiens
MEWTWVFLFLLSVTAGVHSQVHLQQSGAELMKPGASVKISCKATGYTFSSYWIEWVKQRPGHGREWIGEILPGSGSTKYNEKFRGKATFAADTSSNTAYVQLSSLTSEDSAVYYCARLKGYYGGGAMDYWGQGTSVTVSS

SEQ ID NO: 26 - protein, 127 aa, Homo sapiens
MRFQVQVLGLLLLWISGAQCDVQITQSPSYLAASPGETITINCRASKSISKYLAWYQEKPGKTNELLIYSGSTLQSGIPSRFSGSGSGTDFTLTISSLEPEDFAMYYCQQHNEYPWTFGGGTKLEIK

SEQ ID NO: 27 - protein, 5 aa, Homo sapiens
DFYMY

SEQ ID NO: 28 - protein, 17 aa, Homo sapiens
TISDGGSHTYYPDSVKG

SEQ ID NO: 29 - protein, 8 aa, Homo sapiens
DTTIITPY

SEQ ID NO: 30 - protein, 14 aa, Homo sapiens
RSSTGAVTTSNYAN

SEQ ID NO: 31 - protein, 7 aa, Homo sapiens
GTNNRAP

SEQ ID NO: 32 - protein, 9 aa, Homo sapiens
GLWYSNHWV

SEQ ID NO: 33 - protein, 136 aa, Homo sapiens
MNFGLSLIFLVLVLKGVQCEVQLVESGGDLVKPGGSLKLSCAASGFTFSDFYMYWVRRTPEKRLEWVATISDGGSHTYYPDSVKGRFTISRDNAKNNLYLQMRSLKSEDTAMYYCGRDTTIITPYWGQGTLVTVSA

SEQ ID NO: 34 - protein, 128 aa, Homo sapiens
MAWISLILSLLALSSGAISQSVVTQESALTTSPGETVTLTCRSSTGAVTTSNYANWVQEKPDHLFTGLIGGTNNRAPGVPARFSGSLIGDKAALTITGAQTEDEAIYFCGLWYSNHWVFGGGTKLTVL

SEQ ID NO: 35 - protein, 5 aa, Homo sapiens
GYWMT

SEQ ID NO: 36 - protein, 17 aa, Homo sapiens
EINPDSSTINYTPSLRD

SEQ ID NO: 37 - protein, 7 aa, Homo sapiens
GSYYPSY

SEQ ID NO: 38 - protein, 15 aa, Homo sapiens
RASKSVSSSGYSYMN

SEQ ID NO: 39 - protein, 7 aa, Homo sapiens
LASNLES

SEQ ID NO: 40 - protein, 9 aa, Homo sapiens
QHSRELPHT

SEQ ID NO: 41 - protein, 134 aa, Homo sapiens
MDFGLIFFIVALLKGVQCEVKLLESGGGLVQPGGSLKLSCAASGFDFSGYWMTWVRQAPGKGLEWIGEINPDSSTINYTPSLRDKFIISRDNAKNTLYLQMSKVRSEDTALYFCARGSYYPSYWGQGTLVTVSA

SEQ ID NO: 42 - protein, 131 aa, Homo sapiens
METDTLLLWVLLLWVPGSTGDIVLTQSPASLAVSLGQRATISCRASKSVSSSGYSYMNWYQQKPGQPPKLLIYLASNLESGVPARFSGSGSGTDFTLNIHPVEDEDAATYYCQHSRELPHTFGGGTKLEIK

SEQ ID NO: 43 - protein, 5 aa, Homo sapiens
GYWMT

SEQ ID NO: 44 - protein, 17 aa, Homo sapiens
EINPDSSTINYTPSLRD

SEQ ID NO: 45 - protein, 7 aa, Homo sapiens
GSYYPSY

SEQ ID NO: 46 - protein, 15 aa, Homo sapiens
RASKSVSSSGYSYMN

SEQ ID NO: 47 - protein, 7 aa, Homo sapiens
LASNLES

SEQ ID NO: 48 - protein, 9 aa, Homo sapiens
QHSRELPHT

SEQ ID NO: 49 - protein, 134 aa, Homo sapiens; identical to SEQ ID NO: 41

SEQ ID NO: 50 - protein, 131 aa, Homo sapiens; identical to SEQ ID NO: 42

SEQ ID NO: 51 - protein, 6 aa, Homo sapiens
SGYYWN

SEQ ID NO: 52 - protein, 16 aa, Homo sapiens
YISYDGNNNYNPSLKN

SEQ ID NO: 53 - protein, 12 aa, Homo sapiens
PLYDGYYWYFDV

SEQ ID NO: 54 - protein, 11 aa, Homo sapiens
ITSTDIDDDMN

SEQ ID NO: 55 - protein, 7 aa, Homo sapiens
EANTLRP

SEQ ID NO: 56 - protein, 9 aa, Homo sapiens
LQSDNLPYT

SEQ ID NO: 57 - protein, 139 aa, Homo sapiens
MKVLSLLYLLTAIPGILSDVQLQESGPGLVKPSQSLSLTCSVTGYSITSGYYWNWIRQFPGNKLEWMGYISYDGNNNYNPSLKNRISITRDTSKNQFFLKLNSVTTEDTATYYCASPLYDGYYWYFDVWGAGTTVTVSS

SEQ ID NO: 58 - protein, 129 aa, Homo sapiens
MTMFSLALLLSLLLLCVSDSRAETTVTQSPASLSLAIGEKVTIRCITSTDIDDDMNWYQQKPGEPPKLLISEANTLRPGVPSRFSSSGRGTDFVFTIENMLSEDVADYYCLQSDNLPYTFGGGTKLEIK

SEQ ID NO: 59 - DNA, 15 bp, Homo sapiens
  source 1..15: mol_type = unassigned DNA
agctatgatataaac

SEQ ID NO: 60 - DNA, 51 bp, Homo sapiens
  source 1..51: mol_type = unassigned DNA
tggatttttcctggagatgatagtattattcagaatgagaagttcaagggc

SEQ ID NO: 61 - DNA, 27 bp, Homo sapiens
  source 1..27: mol_type = unassigned DNA
ttgggcccattacgagggtttacttac

SEQ ID NO: 62 - DNA, 45 bp, Homo sapiens
  source 1..45: mol_type = unassigned DNA
agagccagtgaaagtgttgatcgttatggcagtagttttatgcac

SEQ ID NO: 63 - DNA, 21 bp, Homo sapiens
  source 1..21: mol_type = unassigned DNA
cgtgcatccaacctagaatct

SEQ ID NO: 64 - DNA, 27 bp, Homo sapiens
  source 1..27: mol_type = unassigned DNA
cagcaaagtaatgaggatccgtggacg

SEQ ID NO: 65 - DNA, 456 bp, Homo sapiens
  source 1..456: mol_type = unassigned DNA
atgggatggagctgggtctttctcttcctcctgtcagtaactgcaggtgtccactcccaggttcagctgcagcagtctggagctgaactggtaaagcctggggcttcagtgaagttgtcctgcaaggcttctggctacatcttcacaagctatgatataaactgggtgaggcagaggcctgaacagggacttgagtggattggatggatttttcctggagatgatagtattattcagaatgagaagttcaagggcaaggccacactgactacagacaaatcctccagcacagtctacatgcagctcagcaggctgacatctgaggactctgctgtctatttctgtgcaagattgggcccattacgagggtttacttactggggccaagggactctggtcactgtctctgcagggtttacttactggggccaagggactctggtcactgtctctgca

SEQ ID NO: 66 - DNA, 393 bp, Homo sapiens
  source 1..393: mol_type = unassigned DNA
atggagacagacacactcctgctatgggtgctgctgctctgggttccaggttccacaggtgacattgtgctgacccaatctccagcttctttggctgtgtctctagggcagagggccaccatatcctgcagagccagtgaaagtgttgatcgttatggcagtagttttatgcactggtaccagcagaaaccaggacagccacccaaactcctcatctatcgtgcatccaacctagaatctgggatccctgccaggttcagtggcagtgggtctaggacagacttcaccctcaccattaatcctgtggaggctgatgatgttgcaacctattactgtcagcaaagtaatgaggatccgtggacgttcggtggaggcaccaagctggaaatcaaa

SEQ ID NO: 67 - DNA, 15 bp, Homo sapiens
  source 1..15: mol_type = unassigned DNA
gactattcaatgaac

SEQ ID NO: 68 - DNA, 51 bp, Homo sapiens
  source 1..51: mol_type = unassigned DNA
aggatcaacactgagactggtgagccaacatatgcagatgacttcaaggga

SEQ ID NO: 69 - DNA, 15 bp, Homo sapiens
  source 1..15: mol_type = unassigned DNA
gactacgctaagcgg

SEQ ID NO: 70 - DNA, 33 bp, Homo sapiens
  source 1..33: mol_type = unassigned DNA
aaggccagtcaggatgtgattactgctgtagcc

SEQ ID NO: 71 - DNA, 21 bp, Homo sapiens
  source 1..21: mol_type = unassigned DNA
tcgacatcctaccggtacact

SEQ ID NO: 72 - DNA, 27 bp, Homo sapiens
  source 1..27: mol_type = unassigned DNA
ctgcaacattatactactccgtggacg

SEQ ID NO: 73 - DNA, 399 bp, Homo sapiens
  source 1..399: mol_type = unassigned DNA
atggcttgggtgtggaccttgctattcctgatggcagctgcccaaagtatccaagcacagatccagttggtacagtctggacctgagctgaagaagcctggagagacagtcaagatctcctgcaaggcttctggttataccttcacagactattcaatgaactgggtgaaacaggctccaggaaagggtttaaagtgggtgggcaggatcaacactgagactggtgagccaacatatgcagatgacttcaagggacggtttgccttctctttggaaacctctgccagcactgcctatttgcagatcaaaaacctcaaaaatgaggacacggctacatatttctgtgttagagactacgctaagcggtggggtcaaggaacctcagtcaccgtctcctca

SEQ ID NO: 74 - DNA, 393 bp, Homo sapiens
  source 1..393: mol_type = unassigned DNA
atgggcatcaaaatggagtcacagattcaggtctttgtattcgtgtctctctggttgtctggtgttgacggagacattgtgatgacccagtctcacaaattcatgtccacatcagtaggagacagggtcagcatcacctgcaaggccagtcaggatgtgattactgctgtagcctggtatcaacagaaaccaggacaatctcctaaactactgatttactcgacatcctaccggtacactggagtccctgatcgcttcactggcagtggatctgggacggatttcactttcaccatcagcagtgtgcaggctgaagacctggcagtttattactgtctgcaacattatactactccgtggacgttcggtggaggcaccaagctggaaatcaaa

SEQ ID NO: 75 - DNA, 15 bp, Homo sapiens
  source 1..15: mol_type = unassigned DNA
agctactggatagag

SEQ ID NO: 76 - DNA, 51 bp, Homo sapiens
  source 1..51: mol_type = unassigned DNA
gagattttacctggaagtggaagtactaagtacaatgagaagtttaggggc

SEQ ID NO: 77 - DNA, 36 bp, Homo sapiens
  source 1..36: mol_type = unassigned DNA
ttgaagggttactacggaggaggtgctatggactac

SEQ ID NO: 78 - DNA, 33 bp, Homo sapiens
  source 1..33: mol_type = unassigned DNA
agggcaagtaagagcattagcaaatatttagcc

SEQ ID NO: 79 - DNA, 21 bp, Homo sapiens
  source 1..21: mol_type = unassigned DNA
tctggatccactttgcaatct

SEQ ID NO: 80 - DNA, 27 bp, Homo sapiens
  source 1..27: mol_type = unassigned DNA
caacagcataatgaatacccgtggacg

SEQ ID NO: 81 - DNA, 420 bp, Homo sapiens
  source 1..420: mol_type = unassigned DNA
atggaatggacctgggtctttctcttcctcctgtcagtaactgcaggtgtccactcccaggttcacctgcagcagtctggagctgagctgatgaagcctggggcctcagtgaaaatatcctgcaaggctactggctacacattcagtagctactggatagagtgggtaaagcagaggcctggacatggccgtgagtggattggagagattttacctggaagtggaagtactaagtacaatgagaagtttaggggcaaggccacattcgctgcagatacatcctccaacacagcctacgtgcaactcagcagcctgacatctgaggactctgccgtctattactgtgcaagattgaagggttactacggaggaggtgctatggactactggggtcaaggaacctcagttaccgtctcttca

SEQ ID NO: 82 - DNA, 381 bp, Homo sapiens
  source 1..381: mol_type = unassigned DNA
atgaggttccaggttcaggttctggggctccttctgctctggatatcaggtgcccagtgtgatgtccagataacccagtctccatcttatcttgctgcatctcctggagaaaccattactattaattgcagggcaagtaagagcattagcaaatatttagcctggtatcaagagaaacctgggaaaactaatgagcttcttatctactctggatccactttgcaatctggaattccatcaaggttcagtggcagtggatctggtacagatttcactctcaccatcagtagcctggagcctgaagattttgcaatgtattactgtcaacagcataatgaatacccgtggacgttcggtggaggcaccaagctggaaatcaaa

SEQ ID NO: 83 - DNA, 15 bp, Homo sapiens
  source 1..15: mol_type = unassigned DNA
gacttttacatgtat

SEQ ID NO: 84 - DNA, 51 bp, Homo sapiens
  source 1..51: mol_type = unassigned DNA
accattagtgatggtggtagtcacacctactatccagacagtgtgaagggg

SEQ ID NO: 85 - DNA, 24 bp, Homo sapiens
  source 1..24: mol_type = unassigned DNA
gatactacgataattactccttac

SEQ ID NO: 86 - DNA, 42 bp, Homo sapiens
  source 1..42: mol_type = unassigned DNA
cgctcaagtactggggctgttacaactagtaactatgccaac

SEQ ID NO: 87 - DNA, 21 bp, Homo sapiens
  source 1..21: mol_type = unassigned DNA
ggtaccaacaaccgagctcca

SEQ ID NO: 88 - DNA, 27 bp, Homo sapiens
  source 1..27: mol_type = unassigned DNA
ggtctttggtacagcaaccattgggtg

SEQ ID NO: 89 - DNA, 408 bp, Homo sapiens
  source 1..408: mol_type = unassigned DNA
atgaacttcgggctcagcttgattttccttgtccttgttttaaaaggtgtccagtgtgaagtgcagctggtggagtctgggggagacttagtgaagcctggagggtccctgaaactctcctgtgcagcctctggattcactttcagtgacttttacatgtattgggttcgccggactccggaaaagaggctggagtgggtcgcaaccattagtgatggtggtagtcacacctactatccagacagtgtgaaggggcgattcaccatctccagagacaatgccaagaacaacctctacctacaaatgagaagtctgaagtctgaggacacagccatgtattactgtggaagagatactacgataattactccttactggggccaagggactctggtcactgtctctgca

SEQ ID NO: 90 - DNA, 384 bp, Homo sapiens
  source 1..384: mol_type = unassigned DNA
atggcctggatttcacttatactctctctcctggctctcagctcaggggccatttcccagtctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtggtctttggtacagcaaccattgggtgttcggtggaggaaccaaactgactgtccta

SEQ ID NO: 91 - DNA, 15 bp, Homo sapiens
  source 1..15: mol_type = unassigned DNA
ggatactggatgact

SEQ ID NO: 92 - DNA, 51 bp, Homo sapiens
  source 1..51: mol_type = unassigned DNA
gaaattaatccagatagcagtacgataaactatacgccatctctaagggat

SEQ ID NO: 93 - DNA, 21 bp, Homo sapiens
  source 1..21: mol_type = unassigned DNA
gggagctactatccctcttac

SEQ ID NO: 94 - DNA, 45 bp, Homo sapiens
  source 1..45: mol_type = unassigned DNA
agggccagcaaaagtgtcagttcatctggctatagttatatgaac

SEQ ID NO: 95 - DNA, 21 bp, Homo sapiens
  source 1..21: mol_type = unassigned DNA
cttgcatccaacctagaatct

SEQ ID NO: 96 - DNA, 27 bp, Homo sapiens
  source 1..27: mol_type = unassigned DNA
cagcacagtagggagcttccgcacacg

SEQ ID NO: 97 - DNA, 402 bp, Homo sapiens
  source 1..402: mol_type = unassigned DNA
atggattttgggctgattttttttattgttgctcttttaaaaggggtccagtgtgaagtgaagcttctcgagtctggaggtggcctggtgcagcctggaggatccctgaaactctcctgtgcagcctcaggattcgattttagtggatactggatgacttgggtccggcaggctccagggaaagggctagaatggattggagaaattaatccagatagcagtacgataaactatacgccatctctaagggataaattcatcatctccagagacaacgccaagaatacgctgtacctgcaaatgagcaaagtgagatctgaggacacagccctttatttctgtgcaagagggagctactatccctcttactggggccaagggactctggtcactgtctctgca

SEQ ID NO: 98 - DNA, 393 bp, Homo sapiens
  source 1..393: mol_type = unassigned DNA
atggagacagacacactcctgttatgggtactgctgctctgggttccaggttccactggggacattgtgctgacacagtctcctgcttccttagctgtatctctggggcagagggccaccatctcatgcagggccagcaaaagtgtcagttcatctggctatagttatatgaactggtaccagcagaaaccaggacagccacccaaactcctcatctatcttgcatccaacctagaatctggggtccctgccaggttcagtggcagtgggtctgggacagacttcaccctcaacatccatcctgtggaggatgaggatgctgcaacctattactgtcagcacagtagggagcttccgcacacgttcggaggggggaccaagctggaaataaaa

SEQ ID NO: 99 - DNA, 15 bp, Homo sapiens
  source 1..15: mol_type = unassigned DNA
ggatactggatgact

SEQ ID NO: 100 - DNA, 51 bp, Homo sapiens; identical to SEQ ID NO: 92
  source 1..51: mol_type = unassigned DNA

SEQ ID NO: 101 - DNA, 21 bp, Homo sapiens
  source 1..21: mol_type = unassigned DNA
gggagctactatccctcttac

SEQ ID NO: 102 - DNA, 45 bp, Homo sapiens; identical to SEQ ID NO: 94
  source 1..45: mol_type = unassigned DNA

SEQ ID NO: 103 - DNA, 21 bp, Homo sapiens
  source 1..21: mol_type = unassigned DNA
cttgcatccaacctagaatct

SEQ ID NO: 104 - DNA, 27 bp, Homo sapiens
  source 1..27: mol_type = unassigned DNA
cagcacagtagggagcttccgcacacg

SEQ ID NO: 105 - DNA, 402 bp, Homo sapiens; identical to SEQ ID NO: 97
  source 1..402: mol_type = unassigned DNA

SEQ ID NO: 106 - DNA, 393 bp, Homo sapiens; identical to SEQ ID NO: 98
  source 1..393: mol_type = unassigned DNA

SEQ ID NO: 107 - DNA, 18 bp, Homo sapiens
  source 1..18: mol_type = unassigned DNA
agtggttattactggaac

SEQ ID NO: 108 - DNA, 48 bp, Homo sapiens
  source 1..48: mol_type = unassigned DNA
tacataagctacgacggtaacaataactacaacccatctctcaaaaat

SEQ ID NO: 109 - DNA, 36 bp, Homo sapiens
  source 1..36: mol_type = unassigned DNA
cctctctatgatggttattactggtacttcgatgtc

SEQ ID NO: 110 - DNA, 33 bp, Homo sapiens
  source 1..33: mol_type = unassigned DNA
ataaccagcactgatattgatgatgatatgaac

SEQ ID NO: 111 - DNA, 21 bp, Homo sapiens
  source 1..21: mol_type = unassigned DNA
gaagccaatactcttcgtcct

SEQ ID NO: 112 - DNA, 27 bp, Homo sapiens
  source 1..27: mol_type = unassigned DNA
ttgcaaagtgataacttgccgtacacg

SEQ ID NO: 113 - DNA, 417 bp, Homo sapiens
  source 1..417: mol_type = unassigned DNA
atgaaagtgttgagtctgttgtacctgttgacagccattcctggtatcctgtctgatgtacagcttcaggagtcaggacctggcctcgtgaaaccttctcagtctctgtctctcacctgctctgtcactggctactccatcaccagtggttattactggaactggatccggcagtttccaggaaacaaactggaatggatgggctacataagctacgacggtaacaataactacaacccatctctcaaaaatcgaatctccatcactcgtgacacgtctaagaaccagtttttcctgaagttgaattctgtgactactgaggacacagctacatattactgtgcaagtcctctctatgatggttattactggtacttcgatgtctggggcgcagggaccacggtcaccgtctcctca

SEQ ID NO: 114 - DNA, 387 bp, Homo sapiens
  source 1..387: mol_type = unassigned DNA
atgaccatgttctcactagctcttctcctcagtcttcttctcctctgtgtctctgattctagggcagaaacaactgtgacccagtctccagcatccctgtccctggctataggagaaaaagtcaccatcagatgcataaccagcactgatattgatgatgatatgaactggtaccagcagaagccaggggaacctcctaagctccttatttcagaagccaatactcttcgtcctggagtcccatcccgattctccagcagtggccgtggtacagattttgtttttacaattgaaaacatgctctcagaagatgttgcagattactactgtttgcaaagtgataacttgccgtacacgttcggaggggggaccaagctggaaataaaa

(19) United States
(12) Patent Application Publication
(10) Pub. No.: US 2024/0117041 A1
(43) Pub. Date: Apr. 11, 2024

(54) ANTIBODIES HAVING SPECIFICITY FOR BTN2 AND USES THEREOF

(71) Applicants: IMCHECK THERAPEUTICS SAS, Marseille (FR); INSERM (INSTITUT NATIONAL DE LA SANTÉ ET DE LA RECHERCHE MÉDICALE), Paris (FR); INSTITUT JEAN PAOLI & IRENE CALMETTES, Marseille (FR); UNIVERSITÉ D'AIX-MARSEILLE, Marseille (FR); CENTRE NATIONAL DE LA RECHECHE SCIENTIFIQUE - CNRS, Paris (FR)

(72) Inventors: Daniel OLIVE, Marseille (FR); Christine PASERO, Marseille (FR)

(21) Appl. No.: 18/147,331

(22) Filed: Dec. 28, 2022

Related U.S. Application Data
(63) Continuation of application No. 16/648,886, filed on Mar. 19, 2020, now Pat. No. 11,572,409.

(30) Foreign Application Priority Data
Sep. 21, 2017 (EP) 17306238.1
Nov. 10, 2017 (EP) 17306563.2

(51) Int. Cl. C07K 16/28

(52) U.S. Cl. CPC C07K 16/2803; C07K 2317/21; C07K 2317/24; C07K 2317/92

(57) ABSTRACT

The present invention relates to antibodies having specificity for BTN2 and uses thereof, in particular for the treatment of cancer.

### CROSS-REFERENCE TO RELATED APPLICATIONS

This application is a Continuation Application of U.S. Ser. No. 16/648,886 filed Mar. 19, 2020, now U.S. Pat. No. 11,572,409, which itself was a national stage filing from international application PCT/EP2018/075689 filed Sep. 21, 2018, and this international application claimed priority to European Application 17306238.1 filed Sep. 21, 2017 and European Application 17306563.2 filed Nov. 10, 2017.

### SEQUENCE LISTING

This application includes as the Sequence Listing the complete contents of the XML file “2022-12-28_13500112C1_seqlisting”, created Dec. 28, 2022, containing 108,361 bytes, hereby incorporated by reference.

### FIELD OF THE INVENTION

The present invention relates to antibodies having specificity for BTN2 and uses thereof.

### BACKGROUND

White blood cells are cells of the immune system involved in defending the body against pathogens. In addition to conventional MHC class I-restricted CD8+CTL and NK cells, other unconventional T cells, notably γδ T cells, display the same sensitivity and cytolytic power as NK and T cells. Vγ9/Vδ2 T cells are the main subset of circulating γδ T cells, representing 1-10% of human peripheral T cells.

Vγ9/Vδ2 T cells are important effectors of the immune defence. They lyse directly pathogen infected or abnormal cells. In addition, they regulate immune responses by inducing dendritic cell (DC) maturation as well as the isotypic switching and immunoglobulin production. This important cell platform of the immune system is strictly regulated by surface receptors, chemokines and cytokines. Vγ9/Vδ2 T cells are activated by nonpeptidic phosphorylated isoprenoid pathway metabolites, referred to as phosphoagonists (PAg).

The priming of T cells is modulated by involvement of specialised cells and secretion of chemotactic cytokines. Nowadays, we know that T-cell activation is the result of two synergistic events. The first is the interaction between the receptor of T cell (TCR) and the major histocompatibility complex (MHC) conjugated with processed antigen on the surface of the antigen presenting cells (APC). The second event is a co-stimulatory antigen-independent signal involving B7 molecules. The lack of co-stimulatory signal induces anergy, i.e. the inhibition of T cell proliferation, cytokine secretion and cytotoxic activities. The study of these pathways may provide insight about the triggering of pathologic events, such as autoimmune or lymphoproliferative disorders.

Butyrophilins constitute a family of transmembrane proteins comprising butyrophilin (BTN), BTN-like (BTNL), and selection and upkeep of intraepithelial T cell (SKINT) proteins (Arnett and Viney, 2014). Their extracellular moieties contain IgV and IgC2 domains exhibiting homology to the corresponding domains of B7 co-stimulatory molecules (Arnett and Viney, 2014), and butyrophilins are thus considered to be members of the extended B7 or Ig superfamily.

BTN1A1, the first butyrophilin identified, is required for the formation, secretion, and stabilization of milk fat globules (Ogg et al., 2004). Then, it has been proposed that B7 genes and MHC class I and II genes may have a common ancestral gene and could encode for proteins involved in similar function, such as T cell activation (Rhodes et al., 2001)

Growing evidence subsequently suggested that butyrophilins play diverse roles in the immune system. Functions have been best elucidated for mouse SKINT1 and human BTN3A1, which are not among the conserved family members. SKINT1 drives the thymic differentiation of mouse Vγ5+Vδ1+ T cells (Boyden et al., 2008).

BTN2 subfamily comprises BTN2A1, BTN2A2 and a pseudogene, BTN2A3 in humans. BTN2A1 and BTN2A2 protein isoforms display an IgV and an IgC extracellular domains, a transmembrane domain, and the characteristic intracellular domain B30.2, as for BTN3A1 and BTN3A3, but not BTN3A2. In mouse, BTN2A2 is a single copy gene and ortholog of the human BTN2A2 gene. Recombinant human BTN2A1-Fc protein revealed that a particular glycoform of BTN2A1 binds to a lectin molecule, DC-SIGN, found on dendritic cells (DCs). Binding of BTN2A1 to DC-SIGN is dependent on high-mannose glycosylation of the protein when expressed by tumor cells (Malcherek et al., 2007). To date, however, no clear function has been identified for human BTN2A1/A2 but some experiments have been performed in mice using recombinant Fc proteins.

Smith et al., 2010 showed that recombinant murine BTN2A2-Fc and BTN1A1-Fc bind to activated T cells, suggesting the presence of one or more receptors on these cells. Immobilized BTN2A2-Fc or BTN2A1-Fc proteins, but not MOG-Fc protein, inhibited the proliferation of CD4 and CD8 T cells activated by anti-CD3. Murine BTN1A1 and BTN2A2 also inhibited T cell metabolism, IL-2, and IFN-g secretion.

Amman et al., 2013 found that binding of mouse BTN2A2-Fc to CD3+ primary mouse T cells stimulated with anti-CD3 and anti-CD28 reduced the number of proliferating cells and entry of cells into the cell cycle. Binding of BTN2A2-Fc to anti-CD3− stimulated T cells inhibited CD3ε, Zap70, and subsequent Erk1/2 activation. Murine BTN2A2-Fc also induce Foxp3 expression and Treg differentiation in vitro.

Sarter et al., 2016 showed that Btn2a2−/− mice exhibited enhanced effector CD4+ and CD8+ T cell responses, impaired CD4+ regulatory T cell induction, potentiated antitumor responses, and exacerbated experimental autoimmune encephalomyelitis.

To date, treatment of autoimmune diseases and prevention of transplantation rejection in graft versus host diseases (GVHD) depends on immunosuppressive agents that have serious side effects, or are not always effective. New immunosuppressive agents are therefore desired.

To date, the treatment of autoimmune diseases and the prevention of transplant rejection in graft versus host disease (GvHD) merely depend on immunosuppressive agents. However such immunosuppressive agents may not be always effective and/or have serious side effects.

There is therefore a need to identify new suppressive agents and/or methods to inhibit immune response in a patient in need thereof.

### SUMMARY

The present disclosure relates to antibodies having specificity for BTN2 and uses thereof.

In particular, it is disclosed herein an antibody which binds to BTN2 (e.g. the human BTN2A1 and BTN2A2 polypeptides) and exhibiting at least one of the following properties:
it inhibits the production of IFN-γ and/or TNF-α by activated Vγ9/Vδ2 T cells, and/or
it inhibits the cytolytic function of activated Vγ9/Vδ2 T cells, and/or
it inhibits the proliferation of activated Vγ9/Vδ2 T cells

In specific embodiments, the anti-BTN2 antibodies according to the present disclosure have specificity for both human butyrophilin-2A1 (BTN2A1) and human butyrophilin-2A2 (BTN2A2).

In specific embodiments, the anti-BTN2 antibodies according to the present disclosure compete for binding to BTN2A2 with at least one of the following reference murine antibodies:
i. mAb 4.15 as obtainable by the hybridoma deposited at the CNCM under deposit number CNCM I-5231;
ii. mAb 5.28 as obtainable by the hybridoma deposited at the CNCM under deposit number CNCM I-5232;
iii. mAb 7.28 as obtainable by the hybridoma deposited at the CNCM under deposit number CNCM I-5233;
iv. mAb 7.48 as obtainable by the hybridoma deposited at the CNCM under deposit number CNCM I-5234;
v. mAb 8.15 as obtainable by the hybridoma deposited at the CNCM under deposit number CNCM I-5235; or
vi. mAb 8.16 as obtainable by the hybridoma deposited at the CNCM under deposit number CNCM I-5236.

In specific embodiments, an anti-BTN2 antibody according to the present disclosure comprises either,
i. a heavy chain and a light chain comprising the 6 CDRs of the antibody mAb 4.15, said mAb 4.15 being obtainable by the hybridoma deposited at the CNCM under deposit number CNCM I-5231;
ii. a heavy chain and a light chain comprising the 6 CDRs of the antibody mAb 5.28, said mAb 5.28 being obtainable by the hybridoma deposited at the CNCM under deposit number CNCM I-5232;
iii. a heavy chain and a light chain comprising the 6 CDRs of the antibody mAb 7.28, said mAb 7.28 being obtainable by the hybridoma deposited at the CNCM under deposit number CNCM I-5233;
iv. a heavy chain and a light chain comprising the 6 CDRs of the antibody mAb 7.48, said mAb 7.48 being obtainable by the hybridoma deposited at the CNCM under deposit number CNCM I-5234;
v. a heavy chain and a light chain comprising the 6 CDRs of the antibody mAb 8.15, said mAb 8.15 being obtainable by the hybridoma deposited at the CNCM under deposit number CNCM I-5235; or,
vi. a heavy chain and a light chain comprising the 6 CDRs of the antibody mAb 8.16, said mAb 8.16 being obtainable by the hybridoma deposited at the CNCM under deposit number CNCM I-5236.

In other specific embodiments, the anti-BTN2 antibody of the present disclosure comprises either,
(i) the H-CDR1, H-CDR2, HCDR3, L-CDR1, L-CDR2 and L-CDR3 of the mAb 4.15 of SEQ ID NOs:3-8 respectively;
(ii) the H-CDR1, H-CDR2, HCDR3, L-CDR1, L-CDR2 and L-CDR3 of the mAb 5.28 of SEQ ID NOs:11-16 respectively;
(iii) the H-CDR1, H-CDR2, HCDR3, L-CDR1, L-CDR2 and L-CDR3 of the mAb 7.28 of SEQ ID NOs:19-24 respectively;
(iv) the H-CDR1, H-CDR2, HCDR3, L-CDR1, L-CDR2 and L-CDR3 of the mAb 7.48 of SEQ ID NOs:27-32 respectively; or,
(v) the H-CDR1, H-CDR2, HCDR3, L-CDR1, L-CDR2 and L-CDR3 of the mAb 8.15 of SEQ ID NOs:35-40 respectively
(vi) the H-CDR1, H-CDR2, HCDR3, L-CDR1, L-CDR2 and L-CDR3 of the mAb 8.16 of SEQ ID NOs:43-48 respectively.

In other specific embodiments, that may be combined with the previous embodiments, the anti-BTN2 antibody of the disclosure is an antibody comprising either,
(i) a heavy chain wherein the VH region has at least 95% identity with SEQ ID NO:9 and a light chain wherein the VL region has at least 95% identity with SEQ ID NO:10;
(ii) a heavy chain wherein the VH region has at least 95% identity with SEQ ID NO:17 and a light chain wherein the VL region has at least 95% identity with SEQ ID NO:18;
(iii) a heavy chain wherein the VH region has at least 95% identity with SEQ ID NO:25 and a light chain wherein the VL region has at least 95% identity with SEQ ID NO:26;
(iv) a heavy chain wherein the VH region has at least 95% identity with SEQ ID NO:33 and a light chain wherein the VL region has at least 95% identity with SEQ ID NO:34;
(v) a heavy chain wherein the VH region has at least 95% identity with SEQ ID NO:41 and a light chain wherein the VL region has at least 95% identity with SEQ ID NO:42; or,
(vi) a heavy chain wherein the VH region has at least 95% identity with SEQ ID NO:49 and a light chain wherein the VL region has at least 95% identity with SEQ ID NO:50.

In specific embodiments, the anti-BTN2 antibody according to the present disclosure does not cross-react with human CD277, in particular it does not cross-react with any one of human BTN3A1, BTN3A2 and BTN3A3.

In specific embodiments, the anti-BTN2 antibody according to the present disclosure inhibits the cytolytic function of activated Vγ9/Vδ2 T cells in the presence of the agonist anti-CD277 antibody mAb20.1.

In specific embodiments, the anti-BTN2 antibody according to the present disclosure inhibits the cytolytic function of activated Vγ9/Vδ2 T cells, for example as activated by co-culture with target cell line (ie Daudi cell line), and/or by phosphoagonists (PAg), and/or by agents that induce the production of phosphoagonists (PAg).

In specific embodiments, said anti-BTN2 antibody is a human, chimeric or humanized antibody.

Another aspect of the present disclosure relates to a nucleic acid molecule which encodes a heavy chain and/or a light chain of any of the anti-BTN2 antibodies as described above.

The disclosure also pertains to a host cell comprising such nucleic acids, in particular for use in the manufacturing of any one of the anti-BTN2 antibodies as described above.

Another aspect of the disclosure relates to the anti-BTN2 antibody as defined above, for use in therapy, for example in a method for treating autoimmune and inflammatory disorders and transplant rejection.

Another aspect of the disclosure relates to a method of treating autoimmune and inflammatory disorders and transplant rejection, in a subject in need thereof comprising administering to the subject, a therapeutically effective amount of the anti-BTN2 antibody as defined above.

Typically, said autoimmune and inflammatory disorders is selected among the group consisting of: rheumatoid arthritis (RA), insulin-dependent diabetes mellitus (Type 1 diabetes), multiple sclerosis (MS), Crohn's disease, systemic lupus erythematosus (SLE), scleroderma, Sjogren's syndrome, pemphigus vulgaris, pemphigoid, Addison's disease, ankylosing spondylitis, aplastic anemia, autoimmune hemolytic anemia, autoimmune hepatitis, coeliac disease, dermatomyositis, Goodpasture's syndrome, Graves' disease, Guillain-Barre syndrome, Hashimoto's disease, idiopathic leucopenia, idiopathic thrombocytopenic purpura, male infertility, mixed connective tissue disease, myasthenia gravis, pernicious anemia, phacogenic uveitis, primary biliary cirrhosis, primary myxoedema, Reiter's syndrome, stiff man syndrome, thyrotoxicosis, ulcerative colitis, and Wegener's granulomatosis.

The disclosure also relates to a pharmaceutical composition comprising the anti-BTN2 antibody as defined above.

The disclosure further provides a method for inhibiting an immune response in a subject, comprising administering to the subject an effective amount of an anti-BTN2 antibody as disclosed herein.

### DETAILED DESCRIPTION OF THE INVENTION

### Definitions

As used herein the term “BTN2” has its general meaning in the art and refers to human BTN2 polypeptides including either BTN2A1 of SEQ ID NO:1 or BTN2A2 of SEQ ID NO:2.

SEQ ID NO: 1: BTN2A isoform 1 precursor (Homo
sapiens):
MESAAALHFSRPASLLLLLLSLCALVSAQFIVVGPTDPILATVGENTTLR
CHLSPEKNAEDMEVRWFRSQFSPAVFVYKGGRERTEEQMEEYRGRTTFVS
KDISRGSVALVIHNITAQENGTYRCYFQEGRSYDEAILHLVVAGLGSKPL
ISMRGHEDGGIRLECISRGWYPKPLTVWRDPYGGVAPALKEVSMPDADGL
FMVTTAVIIRDKSVRNMSCSINNTLLGQKKESVIFIPESFMPSVSPCAVA
LPIIVVILMIPIAVCIYWINKLQKEKKILSGEKEFERETREIALKELEKE
RVQKEEELQVKEKLQEELRWRRTFLHAVDVVLDPDTAHPDLFLSEDRRSV
RRCPFRHLGESVPDNPERFDSQPCVLGRESEASGKHYWEVEVENVIEWTV
GVCRDSVERKGEVLLIPQNGFWTLEMHKGQYRAVSSPDRILPLKESLCRV
GVFLDYEAGDVSFYNMRDRSHIYTCPRSAFSVPVRPFFRLGCEDSPIFIC
PALTGANGVTVPEEGLTLHRVGTHQSL
SEQ ID NO: 2: BTN2A isofom 2 precursor (Homo
sapiens):
MEPAAALHFSLPASLLLLLLLLLLSLCALVSAQFTVVGPANPILAMVGEN
TTLRCHLSPEKNAEDMEVRWFRSQFSPAVFVYKGGRERTEEQMEEYRGRI
TFVSKDINRGSVALVIHNVTAQENGIYRCYFQEGRSYDEAILRLVVAGLG
SKPLIEIKAQEDGSIWLECISGGWYPEPLTVWRDPYGEVVPALKEVSIAD
ADGLFMVTTAVIIRDKYVRNVSCSVNNTLLGQEKETVIFIPESFMPSASP
WMVALAVILTASPWMVSMTVILAVFIIFMAVSICCIKKLQREKKILSGEK
KVEQEEKEIAQQLQEELRWRRTFLHAADVVLDPDTAHPELFLSEDRRSVR
RGPYRQRVPDNPERFDSQPCVLGWESFASGKHYWEVEVENVMVWTVGVCR
HSVERKGEVLLIPQNGFWTLEMFGNQYRALSSPERILPLKESLCRVGVFL
DYEAGDVSFYNMRDRSHIYTCPRSAFTVPVRPFFRLGSDDSPIFICPALT
GASGVMVPEEGLKLHRVGTHQSL

As used herein the term “antibody” or “immunoglobulin” have the same meaning, and will be used equally in the present invention.

The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments.

In natural antibodies, two heavy chains are linked to each other by disulfide bonds and each heavy chain is linked to a light chain by a disulfide bond. There are two types of light chain, lambda (l) and kappa (κ). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CHI, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR).

The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDRs set from each of a heavy and a light chain V region. Framework Regions (FRs) refer to amino acid sequences interposed between CDRs. According the variable regions of the light and heavy chains typically comprise 4 framework regions and 3 CDRs of the following sequence: FR1-CDR1-FR2-CDR2-FR3-CDR3-FR4.

The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35 (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

In specific embodiments, an antibody provided herein is an antibody fragment, and more particularly any protein including an antigen-binding domain of an antibody as disclosed herein. Antibody fragments include, but are not limited to, Fv, Fab, F(ab′)2, Fab′, dsFv, scFv, sc(Fv)2 and diabodies.

As used herein, the term “specificity” refers to the ability of an antibody to detectably bind an epitope presented on an antigen, such as a BTN2. In some embodiments, it is intended to refer to an antibody or protein that binds to human BTN2A2 as expressed in a cell line, for example HEK293F cell lines as described in the examples, preferably with an EC50 below 50 μg/ml and more preferably below 10 μg/ml and even more preferably below 1 g/ml as determined in the Examples and FIG. 1. In some embodiments, it is intended to refer to an antibody or protein that binds to human BTN2A1 as expressed in a cell line, for example HEK293F cell lines, which have been knocked-out for all isoforms of BTN3 and BTN2 as described in the examples, preferably with an EC50 below 1 μg/ml, for example below 0.1 μg/ml; and/or, it is intended to refer to an antibody or protein that binds to human BTN2A2 as expressed in a cell line, for example HEK293F cell lines which have been knocked-out for all isoforms of BTN3 and BTN2 as described in the examples, preferably with an EC50 below 50 μg/ml, for example below 1 μg/ml or below 0.02 μg/ml. In other embodiments, it binds to an antigen recombinant polypeptide with a K_{D} of 100 nM or less, 10 nM or less, 1nM or less, 100 pM or less, or 10 pM or less.

An antibody that “cross-reacts with an antigen other than BTN2” is intended to refer to an antibody that binds that antigen with a K_{D} of 10 nM or less, 1 nM or less, or 100 pM or less. An antibody that “does not cross-react with a particular antigen” is intended to refer to an antibody that binds to that antigen, with a K_{D} of 100 nM or greater, or a K_{D} of 1 μM or greater, or a K_{D} of 10 μM or greater. In certain embodiments, such antibodies that do not cross-react with the antigen exhibit essentially undetectable binding against these proteins in standard binding assays.

An “isolated antibody”, as used herein, refers to an antibody that is substantially free of other antibodies having different antigenic specificities (e.g., an isolated antibody that specifically binds to BTN2 is substantially free of antibodies that specifically bind to other antigens than BTN2). An isolated antibody that specifically binds to BTN2 may, however, have cross-reactivity to other antigens, such as related BTN2 molecules from other species. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The phrases “an antibody recognizing an antigen” and “an antibody having specificity for an antigen” are used interchangeably herein with the term “an antibody which binds specifically to an antigen”.

The term “K_{assoc}” or “Kₐ”, as used herein, is intended to refer to the association rate of a particular antibody-antigen interaction, whereas the term “K_{d} is” or “K_{d},” as used herein, is intended to refer to the dissociation rate of a particular antibody-antigen interaction.

The term “K_{D}”, as used herein, is intended to refer to the dissociation constant, which is obtained from the ratio of Kₐ to Kₐ (i.e. Kₐ/Kₐ) and is expressed as a molar concentration (M). K_{D} values for antibodies can be determined using methods well established in the art. Preferred methods for determining the K_{D} values of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. A method for determining the K_{D} of an antibody is by using surface plasmon resonance, or using a biosensor system such as a Biacore® system.

Specificity can further be exhibited by, e.g., an about 10:1, about 20:1, about 50:1, about 100:1, 10.000:1 or greater ratio of affinity/avidity in binding to the specific antigen versus nonspecific binding to other irrelevant molecules (in this case the specific antigen is a BTN2 polypeptide). The term “affinity”, as used herein, means the strength of the binding of an antibody to an epitope.

In one aspect, the present disclosure relates to an antibody having specificity for BTN2 characterized in that it has at least one of the following properties:
i. it inhibits the production of IFN-γ and TNF-α by activated Vγ9/Vδ2 T cells, and/or
ii. it inhibits the cytolytic function of activated Vγ9/Vδ2 T cells, and/or
iii. it inhibits the proliferation of activated Vγ9/Vδ2 T cells.

The anti-BTN2 antibodies of the present disclosure having such advantageous properties can be screened among anti-BTN2 antibodies using the cellular assays as described in the Examples and in particular the CD107 degranulation assay on Daudi cell lines.

As used herein, by “inhibiting the production of IFNγ or TNFα”, it is meant that a significant decrease of the production of at least IFNγ or TNFα by activated Vγ9/Vδ2 T cells is observed when compared to control activated Vγ9/Vδ2 T cells (with IgG1 or IgG2a as control), said Vγ9/Vδ2 T cells being activated either by co-culture with target cell line (Daudi cell line) or by phosphoagonists (pAg). Typically, the inhibition of the production of IFNγ or TNFα by activated Vγ9/Vδ2 T cells may be measured in a cellular assay by intracellular labelling with antibodies against IFNγ or TNFα and flow cytometry. Such assay is described in more details in the examples below.

As used herein, by “inhibiting the cytolytic function of activated Vγ9/Vδ2 T cells”, it is meant that a significant decrease of the cytolytic function of activated human Vγ9/Vδ2 T cells is observed when compared to control activated human Vγ9/Vδ2 T cells (with IgG1 or IgG2a as control), said human Vγ9/Vδ2 T cells being activated either by co-culture with target cell line (Daudi cell line) or phosphoagonists (pAg). Typically, the inhibition of the cytolytic function of activated Vγ9/Vδ2 T cells, for example in the presence of the agonist antibody mAb20.1, may be measured according to the measurement of the inhibition of the induction of γδ T cells degranulation against a standard cell line, and CD107 as degranulation marker for detecting positive degranulated γδ T cells. Such assay is described in more details in the examples below.

As used herein, by “inhibiting the proliferation of activated Vγ9/Vδ2 T cells”, it is meant that a significant decrease of the proliferation of activated Vγ9/Vδ2 T cells is observed when compared to the proliferation with Vγ9/Vδ2 T cells activated with IgG1 or IgG2a as control, said Vγ9/Vδ2 T cells being activated either by co-culture with target cell line (Daudi cell line) or by phosphoagonists (pAg). Typically, the proliferation of activated Vγ9/Vδ2 T cells may be measured in a cellular assay by CFSE or Cell Trace violet staining and flow cytometry.

In some embodiments, the antibodies of the present invention inhibits the cytolytic function of activated Vγ9/Vδ2 T cells to a level that is substantially equal or superior to at least one of the reference antibodies: mAb 4.15, mAb 5.28, mAb 7.28, mAb 7.48, mAb 8.15, and mAb 8.16 as described below. In other specific embodiments, the anti-BTN2 antibodies inhibit the cytolytic function of activated Vγ9/V6T cells to a level at least equal or superior to mAb 103.2, said mAb 103.2 being disclosed in WO2012/080351.

In some embodiments, the antibodies of the present invention inhibits the production of at least IFNγ or TNFα by activated Vγ9/Vδ2 T cells to a level that is substantially equal or superior to at least one of the reference antibodies: mAb 4.15, mAb 5.28, mAb 7.28, mAb 7.48, mAb 8.15, and mAb 8.16 as described below. In other specific embodiments, the anti-BTN2 antibodies inhibit the cytolytic function of activated Vγ9/Vδ2 T cells to a level at least equal or superior to mAb 103.2, said mAb 103.2 being disclosed in WO2012/080351.

In specific embodiments, the anti-BTN2 antibodies according to the present disclosure are further characterized in that they inhibit the cytolytic function of activated Vγ9/Vδ2 T cells, even in the presence of an agonist anti-CD277 antibody mAb20.1.

The anti-CD277 antibody mAb 20.1 has been disclosed in WO2012/080351 and this antibody increases the cytolytic function of activated Vγ9/Vδ2 T cells. Typically, the inhibition of the cytolytic function of activated Vγ9/Vδ2 T cells in the presence of mAb20.1 may be measured according to the measurement of the inhibition of the induction of γδ T cells degranulation against a standard cell line, for example using the Daudi cell line as standard cell line, and CD107 as degranulation marker for detecting positive degranulated γδ T cells and using mAb 20.1 for example at a concentration of 10% g/ml. Such assay is also described in more details in the examples below.

Reference Antibodies mAbs 1-6

Antibodies of the invention include the reference murine monoclonal antibodies mAb1-mAb6, as produced by the hybridomas which have been deposited at Collection Nationale de Cultures des Microorganismes (CNCM, Institut Pasteur, 25 rue du Docteur Roux, 75724 Paris Cedex 15, France), in accordance with the terms of the Budapest treaty on Sep. 14, 2017, under the respective deposit numbers as described in the Table 1 below:

TABLE 1
mAb1-mAb6

| Antibody | Clone Name | Deposit numbers |
|---|---|---|
| mAb1 | 4.15 | CNCM I-5231 |
| mAb2 | 5.28 | CNCM I-5232 |
| mAb3 | 7.28 | CNCM I-5233 |
| mAb4 | 7.48 | CNCM I-5234 |
| mAb5 | 8.15 | CNCM I-5235 |
| mAb6 | 8.16 | CNCM I-5236 |

The invention further relates to any antibodies comprising the respective VH and VL regions of any one of the above reference antibodies.

The present invention further relates to the hybridomas accessible at the CNCM under deposit numbers CNCM I-5231, CNCM I-5232, CNCM I-5233, CNCM I-5234, CNCM I-5235, or CNCM I-5236.

Other antibodies of the invention include those having amino acids that have been mutated by amino acid deletion, insertion or substitution, yet have at least 60, 70, 80, 90, 95 or 100 percent identity in the CDR regions with the CDR regions of any one of the above reference antibodies.

In some embodiments, the antibody of the invention is a mutant variant of any one of mAb1-mAb6, having the 6 CDR regions 100% identical to the corresponding 6 CDR regions of one of reference mAb1-mAb6, and wherein said mutant variant antibody include mutant amino acid sequences wherein no more than 1, 2, 3, 4 or 5 amino acids have been mutated by amino acid deletion, insertion or substitution in the FR1, FR2, FR3 and FR4 regions when compared with the corresponding framework regions of the corresponding reference antibodies.

In particular embodiments, an anti-BTN2 antibody of the invention, preferably a humanized anti-BTN2, comprises either,
i. a heavy chain and a light chain comprising the 6 CDRs of the antibody mAb 4.15, said mAb 4.15 being obtainable by the hybridoma deposited at the CNCM under deposit number CNCM I-5231;
ii. a heavy chain and a light chain comprising the 6 CDRs of the antibody mAb 5.28, said mAb 5.28 being obtainable by the hybridoma deposited at the CNCM under deposit number CNCM I-5232;
iii. a heavy chain and a light chain comprising the 6 CDRs of the antibody mAb 7.28, said mAb 7.28 being obtainable by the hybridoma deposited at the CNCM under deposit number CNCM I-5233;
iv. a heavy chain and a light chain comprising the 6 CDRs of the antibody mAb 7.48, said mAb 7.48 being obtainable by the hybridoma deposited at the CNCM under deposit number CNCM I-5234;
v. a heavy chain and a light chain comprising the 6 CDRs of the antibody mAb 8.15, said mAb 8.15 being obtainable by the hybridoma deposited at the CNCM under deposit number CNCM I-5235; or,
vi. a heavy chain and a light chain comprising the 6 CDRs of the antibody mAb 8.16, said mAb 8.16 being obtainable by the hybridoma deposited at the CNCM under deposit number CNCM I-5236.

Antibodies of the disclosure thus also include the murine anti-BTN2 antibodies isolated and structurally characterized by their variable heavy and light chain amino acid sequences as described in the Table 2 below:

TABLE 2
Variable heavy and light chain amino acid sequences
of murine reference antibodies of the disclosure

| | VH | VL |
|---|---|---|
| Antibody | Amino acid sequence | Amino acid sequence |
| mAb1 | SEQ ID NO: 9 | SEQ ID NO: 10 |
| mAb2 | SEQ ID NO: 17 | SEQ ID NO: 18 |
| mAb3 | SEQ ID NO: 25 | SEQ ID NO: 26 |
| mAb4 | SEQ ID NO: 33 | SEQ ID NO: 34 |
| mAb5 | SEQ ID NO: 41 | SEQ ID NO: 42 |
| mAb6 | SEQ ID NO: 49 | SEQ ID NO: 50 |

The corresponding amino acid and nucleotide coding sequence of the constant isotype regions of IgG4, IgG1 and their mutant versions are well-known in the art.

Examples of the amino acid sequences of the VH CDR1s (also called HCDR1), VH CDR2s (also called HCDR2), VH CDR3s (also called HCDR1), VL CDR1s (also called LCDR1), VL CDR2s (also called LCDR2), VL CDR3s (also called HCDR3) of some antibodies according to the disclosure are shown in Table 3.

In Table 3, the CDR regions of some antibodies of the present disclosure are delineated using the Chothia system (Chothia C, Lesk A M. 1987, J Mol Biol 196, 901-917).

For the ease of reading, the CDR regions are called hereafter HCDR1, HCDR2, HCDR3, LCDR1, LCDR2, LCDR3 respectively.

TABLE 3
CDR regions of reference murine antibodies according to
Chothia definition

| Original | | | | | | |
|---|---|---|---|---|---|---|
| antibody | HCDR1 | HCDR2 | HCDR3 | LCDR1 | LCDR2 | LCDR3 |
| mAb1 | SEQ ID | SEQ ID | SEQ ID | SEQ ID | SEQ ID | SEQ ID |
| | NO: 3 | NO: 4 | NO: 5 | NO: 6 | NO: 7 | NO: 8 |
| mAb2 | SEQ ID | SEQ ID | SEQ ID | SEQ ID | SEQ ID | SEQ ID |
| | NO: 11 | NO: 12 | NO: 13 | NO: 14 | NO: 15 | NO: 16 |
| mAb3 | SEQ ID | SEQ ID | SEQ ID | SEQ ID | SEQ ID | SEQ ID |
| | NO: 19 | NO: 20 | NO: 21 | NO: 22 | NO: 23 | NO: 24 |
| mAb4 | SEQ ID | SEQ ID | SEQ ID | SEQ ID | SEQ ID | SEQ ID |
| | NO: 27 | NO: 28 | NO: 29 | NO: 30 | NO: 31 | NO: 32 |
| mAb5 | SEQ ID | SEQ ID | SEQ ID | SEQ ID | SEQ ID | SEQ ID |
| | NO: 35 | NO: 36 | NO: 37 | NO: 38 | NO: 39 | NO: 40 |
| mAb6 | SEQ ID | SEQ ID | SEQ ID | SEQ ID | SEQ ID | SEQ ID |
| | NO: 43 | NO: 44 | NO: 45 | NO: 46 | NO: 47 | NO: 48 |

In specific embodiments, the isolated anti-BTN2 antibody according to the disclosure comprises either:
(a) a variable heavy chain polypeptide comprising HCDR1 of SEQ ID NO:3, HCDR2 of SEQ ID NO:4, HCDR3 of SEQ ID NO:5 and a variable light chain polypeptide comprising LCDR1 of SEQ ID NO:6, LCDR2 of SEQ ID NO:7 and LCDR3 of SEQ ID NO:8;
(b) a variable heavy chain polypeptide comprising HCDR1 of SEQ ID NO:11, HCDR2 of SEQ ID NO:12, HCDR3 of SEQ ID NO:13 and a variable light chain polypeptide comprising LCDR1 of SEQ ID NO:14, LCDR2 of SEQ ID NO:15 and LCDR3 of SEQ ID NO:16;
(c) a variable heavy chain polypeptide comprising HCDR1 of SEQ ID NO:19, HCDR2 of SEQ ID NO:20, HCDR3 of SEQ ID NO:21 and a variable light chain polypeptide comprising LCDR1 of SEQ ID NO:22, LCDR2 of SEQ ID NO:23 and LCDR3 of SEQ ID NO:24;
(d) a variable heavy chain polypeptide comprising HCDR1 of SEQ ID NO:27, HCDR2 of SEQ ID NO:28, HCDR3 of SEQ ID NO:29 and a variable light chain polypeptide comprising LCDR1 of SEQ ID NO:30, LCDR2 of SEQ ID NO:31 and LCDR3 of SEQ ID NO:32;
(e) a variable heavy chain polypeptide comprising HCDR1 of SEQ ID NO:35, HCDR2 of SEQ ID NO:36, HCDR3 of SEQ ID NO:37 and a variable light chain polypeptide comprising LCDR1 of SEQ ID NO:38, LCDR2 of SEQ ID NO:39 and LCDR3 of SEQ ID NO:40; or
(f) a variable heavy chain polypeptide comprising HCDR1 of SEQ ID NO:43, HCDR2 of SEQ ID NO:44, HCDR3 of SEQ ID NO:45 and a variable light chain polypeptide comprising LCDR1 of SEQ ID NO:46, LCDR2 of SEQ ID NO:47 and LCDR3 of SEQ ID NO:48.
   wherein said anti-BTN2 antibody has specificity for BTN2.

In other specific embodiments, the isolated anti-BTN2 antibody according to the disclosure comprises either:
(a) a variable heavy chain polypeptide comprising VH of SEQ ID NO:9 and a variable light chain polypeptide VL of SEQ ID NO:10;
(b) a variable heavy chain polypeptide comprising VH of SEQ ID NO:17 and a variable light chain polypeptide VL of SEQ ID NO:18;
(c) a variable heavy chain polypeptide comprising VH of SEQ ID NO:25 and a variable light chain polypeptide VL of SEQ ID NO:26;
(d) a variable heavy chain polypeptide comprising VH of SEQ ID NO:33 and a variable light chain polypeptide VL of SEQ ID NO:34;
(e) a variable heavy chain polypeptide comprising VH of SEQ ID NO:41 and a variable light chain polypeptide VL of SEQ ID NO:42;
(f) a variable heavy chain polypeptide comprising VH of SEQ ID NO:49 and a variable light chain polypeptide VL of SEQ ID NO:50; or,
   wherein said anti-BTN2 antibody has specificity for BTN2.

### Functional Variant Antibodies

In yet another embodiment, a functional variant antibody of the invention has full length heavy and light chain amino acid sequences; or variable region heavy and light chain amino acid sequences, or all 6 CDR regions amino acid sequences that are homologous or preferably identical to the corresponding amino acid sequences of the antibodies mAb1-mAb6 described above, in particular in Tables 1, 2 and 3, and wherein such functional variant antibodies retain the desired functional properties of the original mAb1-mAb6 antibodies.

A functional variant of a VL, VH, or CDR used in the context of a monoclonal antibody of the present invention still allows the antibody to retain at least a substantial proportion (at least about 50%, 60%, 70%, 80%, 90%, 95% or 100%) of the affinity/avidity and/or the specificity/selectivity of the parent antibody (i.e. any one of mAb1-mAb6 antibody) and in some cases such a monoclonal antibody of the present invention may be associated with greater affinity, selectivity and/or specificity than the parent Ab.

Desired functional properties of the original mAb1-mAb6 antibodies may be selected from the group consisting of:
i. it has specificity for BTN2, in particular it binds to human BTN2 as expressed in a cell line, for example HEK293F cell lines expressing human BTN2A2, as described in the examples, preferably with an EC₅₀ below 50 μg/ml and more preferably below 10 μg/ml and even more preferably below 1 g/ml as determined in the Examples and FIG. 1,
ii. it binds to human BTN2A1 as expressed in a cell line, for example HEK293F cell lines, which have been knocked-out for all isoforms of BTN3 and BTN2 as described in the examples, preferably with an EC50 below 1 μg/ml, for example below 0.1 μg/ml,
iii. it binds to human BTN2A2 as expressed in a cell line, for example HEK293F cell lines which have been knocked-out for all isoforms of BTN3 and BTN2 as described in the examples, preferably with an EC50 below 50 μg/ml, for example below 1 μg/ml or below 0.02 μg/ml,
iv. it inhibits production of IFNγ or TNFα of activated Vγ9/Vδ2 T cells,
v. it inhibits the cytolytic function of activated Vγ9/Vδ2 T cells, and/or
vi. it inhibits the proliferation of activated Vγ9/Vδ2 T cells.

For example, the invention relates to functional variant antibodies of mAb1-mAb6, comprising a variable heavy chain (V_{H}) and a variable light chain (V_{L}) sequences where the CDR sequences, i.e. the 6 CDR regions; HCDR1, HCDR2, HCDR3, LCDR1, LCDR2, LCDR3 share at least 60, 70, 90, 95 or 100 percent sequence identity to the corresponding CDR sequences of at least one antibody of mAb1-mAb6, wherein said functional variant antibody specifically binds to BTN2, and the antibody exhibits at least one of the following functional properties:
i. it inhibits production of IFNγ or TNFα of activated Vγ9/Vδ2 T cells,
ii. it inhibits the cytolytic function of activated Vγ9/Vδ2 T cells, and/or,
iii. it inhibits the proliferation of activated Vγ9/Vδ2 T cells.

It further relates to functional variant antibodies of mAb1-mAb6 comprising a heavy chain variable region and a light chain variable region that are at least 80%, 90%, or at least 95% or 100% identical to the corresponding heavy and light chain variable regions of any one of mAb1-mAb6 antibodies, as shown in particular in Table 2; the functional variant antibody specifically binds to BTN2, and exhibits at least one of the following functional properties:
i. it inhibits production of IFNγ or TNFα of activated Vγ9/Vδ2 T cells,
ii. it inhibits the cytolytic function of activated Vγ9/Vδ2 T cells, and/or
iii. it inhibits the proliferation of activated Vγ9/Vδ2 T cells.

In various embodiments, the antibody may exhibit one or two of the desired functional properties discussed above. The antibody can be, for example, a human antibody, a humanized antibody or a chimeric antibody. Preferably the antibody or protein is a humanized human antibody, more preferably a humanized silent antibody.

As used herein, the term “silent” antibody refers to an antibody that exhibits no or low ADCC activity as measured in an in vitro ADCC activity assay measuring cell lysis of target cells.

In one embodiment, the term “no or low ADCC activity” means that the silent antibody exhibit an ADCC activity that is at below 50%, for example below 10% of the ADCC activity that is observed with the corresponding wild type (non silent) antibody for example with a wild type human IgG1 antibody. Preferably, no detectable ADCC activity is observed in an in vitro ADCC activity assay with a silent antibody as compared to a control Fab antibody.

Silenced effector functions can be obtained by mutation in the Fc constant part of the antibodies and have been described in the Art: Strohl 2009 (LALA & N297A); Baudino 2008, D265A (Baudino et al., J. Immunol. 181 (2008): 6664-69, Strohl, CO Biotechnology (2009): 685-91). Examples of silent IgG1 antibodies comprise mutations reducing ADCC at positions 234, 235 and/or 331 in the IgG1 Fc amino acid sequence (EU numbering). Another silent IgG1 antibody comprises the N297A mutation, which results in aglycosylated or non-glycosylated antibodies.

The sequences of CDR variants may differ from the sequence of the CDRs of the parent antibody sequences (as shown for example in Table 3) through mostly conservative substitutions; for instance at least 10, such as at least 9, 8, 7, 6, 5, 4, 3, 2 or 1 of the substitutions in the variant are conservative amino acid residue replacements. In the context of the present invention, conservative substitutions may be defined by substitutions within the classes of amino acids reflected as follows:
Aliphatic residues I, L, V, and M
Cycloalkenyl-associated residues F, H, W, and Y
Hydrophobic residues A, C, F, G, H, I, L, M, R, T, V, W, and Y
Negatively charged residues D and E
Polar residues C, D, E, H, K, N, Q, R, S, and T
Positively charged residues H, K, and R
Small residues A, C, D, G, N, P, S, T, and V
Very small residues A, G, and S
Residues involved in turn A, C, D, E, G, H, K, N, Q, R, S, P, and formation T
Flexible residues Q, T, K, S, G, P, D, E, and R

More conservative substitutions groupings include: valine-leucine-isoleucine, phenylalanine-tyrosine, lysine-arginine, alanine-valine, and asparagine-glutamine. Conservation in terms of hydropathic/hydrophilic properties and residue weight/size also is substantially retained in a variant CDR as compared to a CDR of the any one of mAbs 1-6. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). The retention of similar residues may also or alternatively be measured by a similarity score, as determined by use of a BLAST program (e.g., BLAST 2.2.8 available through the NCBI using standard settings BLOSUM62, Open Gap=11 and Extended Gap=1). Suitable variants typically exhibit at least about 70% of identity to the parent peptide. According to the present invention a first amino acid sequence having at least 70% of identity with a second amino acid sequence means that the first sequence has 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; or 100% of identity with the second amino acid sequence. According to the present invention a first amino acid sequence having at least 50% of identity with a second amino acid sequence means that the first sequence has 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; or 100% of identity with the second amino acid sequence.

In some embodiments, the antibody of the present invention is a chimeric antibody, typically a chimeric mouse/human antibody. The term “chimeric antibody” refers to a monoclonal antibody which comprises a VH domain and a VL domain of an antibody derived from a non-human animal, a CH domain and a CL domain of a human antibody. As the non-human animal, any animal such as mouse, rat, hamster, rabbit or the like can be used. In particular, said mouse/human chimeric antibody may comprise the VH and the VL domains of any one of mAb1-mAb6 reference antibodies.

In some embodiments, the antibody of the present invention is a humanized antibody. In specific embodiments, the antibody of the present invention is a humanized antibody which comprises the 6 CDRs of any one of the mAb1-mAb6 reference antibodies, for example as shown in Table 3. As used herein the term “humanized antibody” refers to antibodies in which the framework regions (FRs) have been modified to comprise the FRs from a donor immunoglobulin of different species (for example human species) as compared to that of the parent immunoglobulin (for example murine CDRs).

In some embodiments, the antibody of the present invention is selected from the group consisting of Fab, F(ab′)2, Fab′ and scFv. As used herein, the term “Fab” denotes an antibody fragment having a molecular weight of about 50,000 and antigen binding activity, in which about a half of the N-terminal side of H chain and the entire L chain, among fragments obtained by treating IgG with a protease, papaine, are bound together through a disulfide bond. The term “F(ab′)2” refers to an antibody fragment having a molecular weight of about 100,000 and antigen binding activity, which is slightly larger than the Fab bound via a disulfide bond of the hinge region, among fragments obtained by treating IgG with a protease, pepsin. The term “Fab′” refers to an antibody fragment having a molecular weight of about 50,000 and antigen binding activity, which is obtained by cutting a disulfide bond of the hinge region of the F(ab′)2. A single chain Fv (“scFv”) polypeptide is a covalently linked VH::VL heterodimer which is usually expressed from a gene fusion including VH and VL encoding genes linked by a peptide-encoding linker. The human scFv fragment of the invention includes CDRs that are held in appropriate conformation, preferably by using gene recombination techniques.

Functional variant antibodies with mutant amino acid sequences can be obtained by mutagenesis (e.g., site-directed or PCR-mediated mutagenesis) of the coding nucleic acid molecules, followed by testing of the encoded altered antibody for retained function (i. e., the functions set forth above) using the functional assays described herein.

Antibodies that Cross-Block any One of mAb1-mAb6 and/or that Bind to the Same Epitope as mAb1-mAb6

Additional antibodies with similar advantageous properties of the reference antibodies mAb1-mAb6 as disclosed herein can be identified based on their ability to cross-compete (e.g., to competitively inhibit the binding of), in a statistically significant manner with any one of the reference antibodies mAb1-mAb6 as described above, in standard BTN2 binding assays.

Test antibody may first be screened for their binding affinity to BTN2, for example from human recombinant antibody libraries using for example phage display technologies or from transgenic mouse expressing human variable region antibodies immunized with BTN2 antigens.

The ability of a test antibody to cross-compete with or inhibit the binding of antibodies of the present invention to human BTN2 demonstrates that the test antibody can compete with that antibody for binding to human BTN2; such an antibody may, according to non-limiting theory, bind to the same or a related (e.g., a structurally similar or spatially proximal) epitope on human BTN2 (e.g. BTN2A2 and/or BTN2A1) as the antibody with which it competes.

To screen an anti-BTN2 antibody for its ability to binds to the same epitope as one of mAb1-mAb6 reference antibodies, for example, HEK293 cells transfected with human BTN2A2 or HEK293 cells knocked-out for all isoforms of BTN2 or BTN3 and expressing human BTN2A2 or human BTN2A1 (as described in the examples) are stained with saturing concentration (10 μg/ml) of one of the reference antibodies mAb1-mAb6 during 30 minutes at 4° C. After 2 washes, different doses of a test anti-BTN2 mAbs are tested (30 min at 4° C.) for their competitive potential with any one of mAb1-mAb6 reference antibodies. The mAbs that do compete for the same binding site as the reference antibody will not be able to recognize BTN2 in the presence of such reference antibodies. The data can be expressed as mean fluorescence intensity.

The selected antibodies can be further tested for the advantageous properties of mAb1-mAb6 in particular with respect to inhibition properties against activated Vγ9Vδ2 T cells.

Accordingly, in one embodiment, the invention provides an isolated antibody which cross-blocks or is cross-blocked by at least one antibody of mAb1-mAb6, from binding to BTN2, wherein said antibody:
i. has specificity to BTN2, in particular it binds to human BTN2 as expressed in a cell line, for example HEK293F cell lines expressing human BTN2A2, as described in the examples, preferably with an EC₅₀ below 50 μg/ml and more preferably below 10 μg/ml and even more preferably below 1 g/ml as determined in the Examples and FIG. 1,
ii. it binds to human BTN2A1 as expressed in a cell line, for example HEK293F cell lines, which have been knocked-out for all isoforms of BTN3 and BTN2 as described in the examples, preferably with an EC50 below 1 μg/ml, for example below 0.1 μg/ml,
iii. it binds to human BTN2A2 as expressed in a cell line, for example HEK293F cell lines which have been knocked-out for all isoforms of BTN3 and BTN2 as described in the examples, preferably with an EC50 below 50 μg/ml, for example below 1 μg/ml or below 0.02 μg/ml,
iv. it inhibits production of IFNγ and/or TNFα by activated Vγ9Vδ2 T cells,
v. it inhibits the cytolytic function of activated Vγ9Vδ2 T cells, and/or
vi. it inhibits the proliferation of activated Vγ9/Vδ2 T cells.

In another embodiment, the invention provides antibodies that bind to the same epitope as do at least one of the anti-BTN2 antibodies mAb1-mAb6 as described herein.

In a certain embodiment, the cross-blocking antibodies or antibody that binds to the same epitope on human BTN2 as any one of mAb1-mAb6, is a chimeric, humanized or human recombinant antibody.

### Generation of Transfectomas Producing Monoclonal Antibodies

The antibodies of the present invention are produced by any techniques known in the art, such as, without limitation, any chemical, biological, genetic or enzymatic technique, either alone or in combination. Typically, knowing the amino acid sequence of the desired sequence, one skilled in the art can readily produce said antibodies, by standard techniques for production of polypeptides. For instance, they can be synthesized using well-known solid phase method, preferably using a commercially available peptide synthesis apparatus (such as that made by Applied Biosystems, Foster City, California) and following the manufacturer's instructions. Alternatively, antibodies of the present invention can be synthesized by recombinant DNA techniques well-known in the art. For example, antibodies can be obtained as DNA expression products after incorporation of DNA sequences encoding the antibodies into expression vectors and introduction of such vectors into suitable eukaryotic or prokaryotic hosts that will express the desired antibodies, from which they can be later isolated using well-known techniques.

Accordingly, a further object of the invention relates to a nucleic acid molecule encoding an antibody according to the invention. More particularly the nucleic acid molecule encodes a heavy chain or a light chain of an antibody of the present invention. More particularly the nucleic acid molecule comprises a VH or VL coding region having at least 70%, 80%, 90%, 95% or 100% of identity to the corresponding nucleic acid encoding heavy chain variable region (VH region) or light chain variable region (VL) of any one of the reference antibodies mAb1-mAb6.

Typically, said nucleic acid is a DNA or RNA molecule, which may be included in any suitable vector, such as a plasmid, cosmid, episome, artificial chromosome, phage or a viral vector. As used herein, the terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. So, a further object of the invention relates to a vector comprising a nucleic acid of the invention. Such vectors may comprise regulatory elements, such as a promoter, enhancer, terminator and the like, to cause or direct expression of said antibody upon administration to a subject. Examples of promoters and enhancers used in the expression vector for animal cell include early promoter and enhancer of SV40, LTR promoter and enhancer of Moloney mouse leukemia virus, promoter and enhancer of immunoglobulin H chain and the like. Any expression vector for animal cell can be used, so long as a gene encoding the human antibody C region can be inserted and expressed. Examples of suitable vectors include pAGE107, pAGE103, pHSG274, pKCR, pSG1 beta d2-4 and the like. Other examples of plasmids include replicating plasmids comprising an origin of replication, or integrative plasmids, such as for instance pUC, pcDNA, pBR, and the like. Other examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

A further object of the present invention relates to a host cell which has been transfected, infected or transformed by a nucleic acid and/or a vector as described above. As used herein, the term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed”.

The nucleic acids of the invention may be used to produce an antibody of the present invention in a suitable expression system. The term “expression system” means a host cell and compatible vector under suitable conditions, e.g. for the expression of a protein coded for by foreign DNA carried by the vector and introduced to the host cell. Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al; 1980), rat YB2/3HL.P2.G11.16Ag.20 cell (ATCC CRL1662, hereinafter referred to as “YB2/0 cell”), and the like.

The present invention also relates to a method of producing a recombinant host cell expressing an antibody according to the invention, said method comprising the steps of: (i) introducing in vitro or ex vivo a recombinant nucleic acid or a vector as described above into a competent host cell, (ii) culturing in vitro or ex vivo the recombinant host cell obtained and (iii), optionally, selecting the cells which express and/or secrete said antibody. Such recombinant host cells can be used for the production of antibodies of the present invention.

Antibodies of the present invention are suitably separated from the culture medium by conventional immunoglobulin purification procedures such as, for example, protein A-Sepharose®, hydroxylapatite chromatography, gel electrophoresis, dialysis, or affinity chromatography.

In some embodiments, the human chimeric antibody of the present invention can be produced by obtaining nucleic sequences encoding VL and VH domains as previously described, constructing a human chimeric antibody expression vector by inserting them into an expression vector for animal cell having genes encoding human antibody CH and human antibody CL, and expressing the coding sequence by introducing the expression vector into an animal cell. As the CH domain of a human chimeric antibody, it may be any region which belongs to human immunoglobulin, but those of IgG class are suitable and any one of subclasses belonging to IgG class, such as IgG1, IgG2, IgG3 and IgG4, can also be used. Also, as the CL of a human chimeric antibody, it may be any region which belongs to Ig, and those of kappa class or lambda class can be used. Methods for producing chimeric antibodies involve conventional recombinant DNA and gene transfection techniques are well known in the art (See Morrison S L. et al. (1984) and patent documents U.S. Pat. Nos. 5,202,238; and 5,204,244).

The humanized antibody of the present invention may be produced by obtaining nucleic acid sequences encoding CDR domains, as previously described, constructing a humanized antibody expression vector by inserting them into an expression vector having genes encoding (i) a heavy chain constant region and heavy chain variable framework regions identical to that of a human antibody and (ii) a light chain constant region and light chain variable framework regions identical to that of a human antibody, and expressing the genes by introducing the expression vector into suitable cell line. The humanized antibody expression vector may be either of a type in which a gene encoding an antibody heavy chain and a gene encoding an antibody light chain exists on separate vectors or of a type in which both genes exist on the same vector (tandem type). In respect of easiness of construction of a humanized antibody expression vector, easiness of introduction into cell lines, and balance between the expression levels of antibody H and L chains in cell lines, humanized antibody expression vector of the tandem type is preferred. Examples of tandem type humanized antibody expression vector include pKANTEX93 (WO 97/10354), pEE18 and the like.

Methods for humanizing antibodies based on conventional recombinant DNA and gene transfection techniques are well known in the art (See, e. g., Riechmann L. et al. 1988; Neuberger M S. et al. 1985). Antibodies can be humanized using a variety of techniques known in the art including, for example, CDR-grafting (EP 239,400; PCT publication WO91/09967; U.S. Pat. Nos. 5,225,539; 5,530,101; and 5,585,089), veneering or resurfacing (EP 592,106; EP 519,596; Padlan E A (1991); Studnicka G M et al. (1994); Roguska M A. et al. (1994)), and chain shuffling (U.S. Pat. No. 5,565,332). The general recombinant DNA technology for preparation of such antibodies is also known (see European Patent Application EP 125023 and International Patent Application WO 96/02576).

The Fab of the present invention can be obtained by treating an antibody which specifically reacts with AMH with a protease, papaine. Also, the Fab can be produced by inserting DNA encoding Fab of the antibody into a vector for prokaryotic expression system, or for eukaryotic expression system, and introducing the vector into a procaryote or eucaryote (as appropriate) to express the Fab.

The F(ab′)2 of the present invention can be obtained treating an antibody which specifically reacts with AMH with a protease, pepsin. Also, the F(ab′)2 can be produced by binding Fab′ described below via a thioether bond or a disulfide bond.

The Fab′ of the present invention can be obtained treating F(ab′)2 which specifically reacts with AMH with a reducing agent, dithiothreitol. Also, the Fab′ can be produced by inserting DNA encoding Fab′ fragment of the antibody into an expression vector for prokaryote, or an expression vector for eukaryote, and introducing the vector into a prokaryote or eukaryote (as appropriate) to perform its expression.

The scFv of the present invention can be produced by obtaining cDNA encoding the VH and VL domains as previously described, constructing DNA encoding scFv, inserting the DNA into an expression vector for prokaryote, or an expression vector for eukaryote, and then introducing the expression vector into a prokaryote or eukaryote (as appropriate) to express the scFv.

To generate a humanized scFv fragment, the well-known technology called CDR grafting may be used, which involves selecting the complementary determining regions (CDRs) from a donor scFv fragment, and grafting them onto a human scFv fragment framework of known three dimensional structure (see, e. g., WO98/45322; WO 87/02671; U.S. Pat. Nos. 5,859,205; 5,585,089; 4,816,567; EP0173494).

Engineered antibodies of the present invention further include those in which modifications have been made to framework residues within VH and/or VL, e.g. to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis. Such “backmutated” antibodies are also intended to be encompassed by the invention. Another type of framework modification involves mutating one or more residues within the framework region, or even within one or more CDR regions, to remove T cell-epitopes to thereby reduce the potential immunogenicity of the antibody. This approach is also referred to as “deimmunization” and is described in further detail in U.S. Patent Publication No. 20030153043 by Carr et al.

### Fc Engineering

The antibody of the invention can be characterized by one or more of the functional or structural features of the aspects described above, or by any combination of selected functional and structural features.

The antibody of the invention may be of any isotype. The choice of isotype typically will be guided by the desired effector functions, such as ADCC silencing. Exemplary isotypes are IgG1, IgG2, IgG3, and IgG4. Either of the human light chain constant regions, kappa or lambda, may be used. If desired, the class of an antibody of the present invention may be switched by known methods. Typical, class switching techniques may be used to convert one IgG subclass to another, for instance from IgG1 to IgG2. Thus, the effector function of the antibodies of the present invention may be changed by isotype switching to, e.g., an IgG1, IgG2, IgG3, IgG4, IgD, IgA, IgE, or IgM antibody for various therapeutic uses. In some embodiments, the antibody of the invention is a full-length antibody. In some embodiments, the full-length antibody is an IgG1 antibody. In some embodiments, the full-length antibody is an IgG4 antibody. In some embodiments, the BTN2-specific IgG4 antibody is a stabilized IgG4 antibody. Examples of suitable stabilized IgG4 antibodies are antibodies wherein arginine at position 409 in a heavy chain constant region of human IgG4, which is indicated in the EU index as in Kabat et al. supra, is substituted with lysine, threonine, methionine, or leucine, preferably lysine (described in WO2006033386) and/or wherein the hinge region comprises a Cys-Pro-Pro-Cys sequence. Other suitable stabilized IgG4 antibodies are disclosed in WO2008145142.

In some embodiments, the antibody of the present invention does not comprise a Fc portion that induces antibody dependent cellular cytotoxicity (ADCC). The terms “Fc domain,” “Fc portion,” and “Fc region” refer to a C-terminal fragment of an antibody heavy chain, e.g., from about amino acid (aa) 230 to about aa 450 of human gamma heavy chain or its counterpart sequence in other types of antibody heavy chains (e.g., a, 6, F and p for human antibodies), or a naturally occurring allotype thereof. Unless otherwise specified, the commonly accepted Kabat amino acid numbering for immunoglobulins is used throughout this disclosure (see Kabat et al. (1991) Sequences of Protein of Immunological Interest, 5th ed., United States Public Health Service, National Institute of Health, Bethesda, MD). In some embodiments, the antibody of the present invention does not comprise an Fc domain capable of substantially binding to a FcgRIIIA (CD16) polypeptide. In some embodiments, the antibody of the present invention lacks an Fc domain (e.g. lacks a CH2 and/or CH3 domain) or comprises an Fc domain of IgG2 or IgG4 isotype. In some embodiments, the antibody of the present invention consists of or comprises a Fab, Fab′, Fab′-SH, F (ab′) 2, Fv, a diabody, single-chain antibody fragment, or a multispecific antibody comprising multiple different antibody fragments. In some embodiments, the antibody of the present invention is not linked to a toxic moiety. In some embodiments, one or more amino acids selected from amino acid residues can be replaced with a different amino acid residue such that the antibody has altered C2q binding and/or reduced or abolished complement dependent cytotoxicity (CDC). This approach is described in further detail in U.S. Pat. Nos. 6,194,551.

Another modification of the antibodies herein that is contemplated by the invention is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (CI-CIO) alkoxy- or aryloxy-poly ethylene glycol or polyethylene glycol-maleimide. In some embodiments, the antibody to be pegylated is an aglycosylated antibody. Methods for pegylating proteins are known in the art and can be applied to the antibodies of the present invention. See for example, EP 0154 316 by Nishimura et al. and EP 0 401 384 by Ishikawa et al.

Another modification of the antibodies that is contemplated by the invention is a conjugate or a protein fusion of at least the antigen-binding region of the antibody of the present invention to serum protein, such as human serum albumin or a fragment thereof to increase half-life of the resulting molecule.

In some embodiments, the invention also provides a multispecific antibody. Exemplary formats for the multispecific antibody molecules of the invention include, but are not limited to (i) two antibodies cross-linked by chemical heteroconjugation, one with a specificity to BTN2 and another with a specificity to a second antigen; (ii) a single antibody that comprises two different antigen-binding regions; (iii) a single-chain antibody that comprises two different antigen-binding regions, e.g., two scFvs linked in tandem by an extra peptide linker; (iv) a dual-variable-domain antibody (DVD-Ig™), where each light chain and heavy chain contains two variable domains in tandem through a short peptide linkage (Wu et al., Generation and Characterization of a Dual Variable Domain Immunoglobulin (DVD-Ig™) Molecule, in: Antibody Engineering, Springer Berlin Heidelberg (2010)); (v) a chemically-linked bispecific (Fab′)2 fragment; (vi) a TandAb, which is a fusion of two single chain diabodies resulting in a tetravalent bispecific antibody that has two binding sites for each of the target antigens; (vii) a flexibody, which is a combination of scFvs with a diabody resulting in a multivalent molecule; (viii) a so called “dock and lock” molecule, based on the “dimerization and docking domain” in Protein Kinase A, which, when applied to Fabs, can yield a trivalent bispecific binding protein consisting of two identical Fab fragments linked to a different Fab fragment; (ix) a so-called Scorpion molecule, comprising, e.g., two scFvs fused to both termini of a human Fab-arm; and (x) a diabody. Another exemplary format for bispecific antibodies is IgG-like molecules with complementary CH3 domains to force heterodimerization. Such molecules can be prepared using known technologies, such as, e.g., those known as Triomab®/Quadroma (Trion Pharma/Fresenius Biotech), Knob-into-Hole (Genentech), CrossMAb (Roche) and electrostatically-matched (Amgen), LUZ-Y (Genentech), Strand Exchange Engineered Domain body (SEEDbody) (EMD Serono), Biclonics® (Merus) and DuoBody® (Genmab A/S) technologies. In some embodiments, the bispecific antibody is obtained or obtainable via a controlled Fab-arm exchange, typically using DuoBody® technology. In vitro methods for producing bispecific antibodies by controlled Fab-arm exchange have been described in WO2008119353 and WO 2011131746 (both by Genmab A/S). In one exemplary method, described in WO 2008119353, a bispecific antibody is formed by “Fab-arm” or “half-molecule” exchange (swapping of a heavy chain and attached light chain) between two monospecific antibodies, both comprising IgG4-like CH3 regions, upon incubation under reducing conditions. The resulting product is a bispecific antibody having two Fab-arms which may comprise different sequences. In another exemplary method, described in WO 2011131746, bispecific antibodies of the present invention are prepared by a method comprising the following steps, wherein at least one of the first and second antibodies is the antibody of the present invention: a) providing a first antibody comprising an Fc region of an immunoglobulin, said Fc region comprising a first CH3 region; b) providing a second antibody comprising an Fc region of an immunoglobulin, said Fc region comprising a second CH3 region; wherein the sequences of said first and second CH3 regions are different and are such that the heterodimeric interaction between said first and second CH3 regions is stronger than each of the homodimeric interactions of said first and second CH3 regions; c) incubating said first antibody together with said second antibody under reducing conditions; and d) obtaining said bispecific antibody, wherein the first antibody is the antibody of the present invention and the second antibody has a different binding specificity, or vice versa. The reducing conditions may, for example, be provided by adding a reducing agent, e.g. selected from 2-mercaptoethylamine, dithiothreitol and tris(2-carboxyethyl)phosphine. Step d) may further comprise restoring the conditions to become non-reducing or less reducing, for example by removal of a reducing agent, e.g. by desalting. Preferably, the sequences of the first and second CH3 regions are different, comprising only a few, fairly conservative, asymmetrical mutations, such that the heterodimeric interaction between said first and second CH3 regions is stronger than each of the homodimeric interactions of said first and second CH3 regions. More details on these interactions and how they can be achieved are provided in WO 2011131746, which is hereby incorporated by reference in its entirety. The following are exemplary embodiments of combinations of such asymmetrical mutations, optionally wherein one or both Fc-regions are of the IgG1 isotype.

### Uses and Methods of the Invention

The antibodies or proteins of the present invention have in vitro and in vivo diagnostic and therapeutic utilities. For example, these molecules can be administered to cells in culture, e.g. in vitro or in vivo, or in a subject, e.g., in vivo, to treat, prevent or diagnose a variety of disorders.

The methods are particularly suitable for treating, preventing or diagnosing BTN2-related disorders and/or autoimmune, inflammatory disorders, and transplant rejection.

The disclosure also pertains to the methods of manufacturing a medicament for use in the treatment of inflammatory conditions, autoimmune diseases and organ or tissue transplant rejection, said medicament comprising an anti-BTN2 antibody of the present disclosure as described in the previous sections.

As used herein, a “BTN2-related disorder” includes conditions associated with or characterized by aberrant BTN2A1 or BTN2A2 levels and/or diseases or conditions that can be treated by modulating BTN2A1 and/or BTN2A2 induced signaling activity in human blood cells e.g. by inhibiting the production of IFNγ or TNFα of activated of Vγ9Vδ2 T cells and/or the cytolytic function of activated Vγ9Vδ2 T cells. These include inflammatory conditions, autoimmune diseases and organ or tissue transplant rejection.

Examples of autoimmune diseases which may be treated include but are not limited to rheumatoid arthritis (RA), insulin-dependent diabetes mellitus (Type 1 diabetes), multiple sclerosis (MS), Crohn's disease, systemic lupus erythematosus (SLE), scleroderma, Sjogren's syndrome, pemphigus vulgaris, pemphigoid, Addison's disease, ankylosing spondylitis, aplastic anemia, autoimmune hemolytic anemia, autoimmune hepatitis, coeliac disease, dermatomyositis, Goodpasture's syndrome, Graves' disease, Guillain-Barre syndrome, Hashimoto's disease, idiopathic leucopenia, idiopathic thrombocytopenic purpura, male infertility, mixed connective tissue disease, myasthenia gravis, pernicious anemia, phacogenic uveitis, primary biliary cirrhosis, primary myxoedema, Reiter's syndrome, stiff man syndrome, thyrotoxicosis, ulcerative colitis, and Wegener's granulomatosis.

The antibodies of the invention may be administered as the sole active ingredient or in conjunction with, e.g. as an adjuvant to or in combination to, other drugs e.g. immunosuppressive or immunomodulating agents or other anti-inflammatory agents, e.g. for the treatment or prevention of diseases mentioned above.

An object of the present invention relates to a method of inhibiting an immune response in a subject, in particular inhibiting the cytolytic property of Vγ9Vδ2 T cells in a subject in need thereof, comprising administering to the subject a therapeutically effective amount of an antibody of the present invention.

As used herein, the term “treatment” or “treat” refer to both prophylactic or preventive treatment as well as curative or disease modifying treatment, including treatment of subject at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “therapeutically effective amount” refers to an amount effective, at dosages and for periods of time necessary, to achieve a desired therapeutic result. A therapeutically effective amount of the antibody of the present invention may vary according to factors such as the disease state, age, sex, and weight of the individual, and the ability of the antibody of the present invention to elicit a desired response in the individual. A therapeutically effective amount is also one in which any toxic or detrimental effects of the antibody or antibody portion are outweighed by the therapeutically beneficial effects. The efficient dosages and dosage regimens for the antibody of the present invention depend on the disease or condition to be treated and may be determined by the persons skilled in the art. A physician having ordinary skill in the art may readily determine and prescribe the effective amount of the pharmaceutical composition required. For example, the physician could start doses of the antibody of the present invention employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. Typically, the ability of a compound to treat autoimmune disorders, for example, be evaluated in an animal model system predictive of efficacy in treating autoimmune disorders. Alternatively, this property of a composition may be evaluated by examining the ability of the compound to inhibit induction of immune response by in vitro assays known to the skilled practitioner. A therapeutically effective amount of a therapeutic compound may decrease immune or inflammatory response, or otherwise ameliorate symptoms in a subject. One of ordinary skill in the art would be able to determine such amounts based on such factors as the subject's size, the severity of the subject's symptoms, and the particular composition or route of administration selected. An exemplary, non-limiting range for a therapeutically effective amount of an antibody of the present invention is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of an antibody of the present invention is 0.02-100 mg/kg, such as about 0.02-30 mg/kg, such as about 0.05-10 mg/kg or 0.1-3 mg/kg, for example about 0.5-2 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. In some embodiments, the efficacy may be monitored by visualization of the disease area, or by other diagnostic methods described further herein, e.g. by performing one or more PET-CT scans, for example using a labeled antibody of the present invention, fragment or mini-antibody derived from the antibody of the present invention. If desired, an effective daily dose of a pharmaceutical composition may be administered as two, three, four, five, six or more sub-doses administered separately at appropriate intervals throughout the day, optionally, in unit dosage forms. In some embodiments, the human monoclonal antibodies of the present invention are administered by slow continuous infusion over a long period, such as more than 24 hours, in order to minimize any unwanted side effects. An effective dose of an antibody of the present invention may also be administered using a weekly, biweekly or triweekly dosing period. The dosing period may be restricted to, e.g., 8 weeks, 12 weeks or until clinical progression has been established. As non-limiting examples, treatment according to the present invention may be provided as a daily dosage of an antibody of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

Typically, the antibody of the present invention is administered to the subject in the form of a pharmaceutical composition which comprises a pharmaceutically acceptable carrier. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a patient, the composition will be formulated for administration to the patient. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m² and 500 mg/m². However, it will be appreciated that these schedules are exemplary and that an optimal schedule and regimen can be adapted taking into account the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g. 1 ml for intramuscular), and between about 1 ng to about 100 mg, e.g. about 50 ng to about 30 mg or more preferably, about 5 mg to about 25 mg, of an anti-BTN2 antibody of the invention.

The invention will be further illustrated by the following figures and examples. However, these examples and figures should not be interpreted in any way as limiting the scope of the present invention.

### LEGENDS OF THE FIGURES

FIG. 1: Binding of BTN2 mAbs on HEK293F cells. HEK293F cells were transfected with BTN2A2-Flag plasmid. At day 1 after transfection, BTN2A2 transfected HEK293F cells (A) and non-transfected HEK293F cells (B) were stained with purified mouse anti-human BTN2 mAbs for 20 min at 4° C. Monoclonal ANTI-FLAG™ antibody serves as positive control for transfection. After 2 washes, cells were incubated with a secondary goat anti-mouse IgG PE for 20 min at 4° C. and a marker for cell viability. Cells were acquired on a LSRFortessa™ (BD) and analyzed with FlowJo™ (TreeStar).

FIG. 2: BTN2 is expressed on cancer cell lines. Cancer cell lines derivated from solid tumors (A), or hematopoietic malignancies (B) were stained with purified BTN2 (8.16) mAb (10 μg/ml) for 20 min at 4° C. After 2 washes, cells were incubated with a secondary goat anti-mouse IgG PE for 20 min at 4° C. and a marker for cell viability. Cells were acquired on a LSRFortessa™ (BD) and analyzed with FlowJo™ (TreeStar). Cell lines were derived from: prostate cancer (PC3, DU145, LNCaP), melanoma (Gerlach), pancreatic cancer (L-IPC, Panc-1, Mia-PACA-2), colorectal cancer (Caco-2), Hela (cervical cancer), renal cancer (A498), lung cancer (A549), breast cancer (MDA-MB-134, MDA-MB-231, SKBR3), Burkitt lymphoma (Daudi, Raji), Non-Hodgkin's lymphoma (RL), chronic myelogenous leukemia (K562), acute myeloid leukemia (U937).

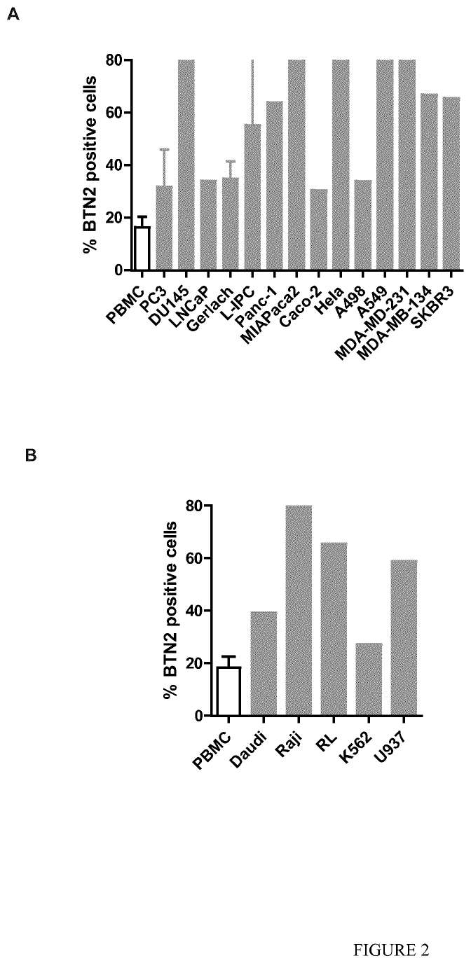

FIG. 3: BTN2 mAbs inhibit the cytolytic function of Vγ9Vδ2 T cells. γδ-T cells were expanded from PBMCs of 6 healthy donors (see Material and Methods). Purified γδ-T cells were simulated overnight in IL-2 (200 UI/ml). Then, γδ-T cells were co-cultured at 37° C. with Daudi target cell line (at effector:target (E:T) ratio of 1:1) with anti-CD107a and anti-CD1072 antibodies and with or without anti-BTN2 (4.15, 5.28, 7.28, 7.48, 8.15, 8.16, 8.33) mAbs (10 μg/ml). After 4 h, cells were collected, fixed and permeabilized, then stained with intracellular mAb (IFN-γ, TNF-α) and analyzed by flow cytometry. The figure shows (A) the degranulation of γδ-T cells, and the production of inflammatory cytokines (B) TNF-α, (C) IFN-γ. Lower dashed line represents the basal cytolytic function of γδ-T cells against Daudi cell line.

FIG. 4: BTN2 mAbs inhibit the agonist effect of anti-CD277 (20.1) mAb on the cytolytic function of Vγ9Vδ2 T cells. Purified γδ-T cells were simulated overnight in IL-2 (200 UI/ml). Then, γδ-T cells were co-cultured at 37° C. with Daudi target cell line (at effector:target (E:T) ratio of 1:1) with anti-CD107a and anti-CD1072 antibodies and Golgi stop, with or without anti-BTN2 (4.15, 5.28, 7.28, 7.48, 8.15, 8.16, 8.33) mAbs in combination or not with anti-CD277 20.1 mAb. After 4 h, cells were collected and analyzed by flow cytometry. The figure shows the percentage of CD107 (degranulation marker) positive cells among γδ-T cells. Lower dashed line represents the basal degranulation of γδ-T cells against Daudi cell line. Upper dashed line represents the median degranulation observed with anti-CD277 20.1 antibody.

FIG. 5: BTN2 mAbs does not cross-react with HEK293F cells transfected with CD277 isoforms (BTN3A1, BTN3A2 or BTN3A3). HEK293F cells transfected with BTN3A1, BTN3A2, BTN3A3 and non-transfected HEK293F cells were stained with purified mouse anti-human BTN2 mAbs, or with mouse anti-human CD277 20.1 mAb, or with isotypic control IgG1 for 20 min at 4° C. ANTI-FLAG™ serves as positive control for transfection. After 2 washes, cells were incubated with a secondary goat anti-mouse IgG PE for 20 min at 4° C. and a marker for cell viability. Cells were acquired on a LSRFortessa™ (BD) and analyzed with FlowJo™ (TreeStar).

### EXAMPLES

### Material and Methods

### Cell Culture

Peripheral blood mononuclear cells (PBMCs) were obtained from healthy volunteer donors (HV) provided by the local Blood Bank (EFS-Marseille-France) and isolated by density gradient (Eurobio).

The Burkitt lymphoma cell line, Daudi, was obtained from the American Type Culture Collection and cultured (0.5×10⁶/mL) in RPMI 1640 medium with 10% FCS.

### Expansion of γδ-T Cells

Effector γδ-T cells were established as previously described. PBMCs from HV were stimulated with Zoledronate (Sigma, 1 μM) and rhIL-2 (Proleukin, 200 IU/mL) at Day 0. From Day 5, rhIL-2 was renewed every two days and cells were kept at 1.15×10⁶/mL for 15 days. The last day, the purity of γδ T cells was evaluated by flow cytometry. Only cells cultures that reached more than 80% of γδ T Cells, were selected to be used in functional tests. Purified γδ-T cells were thawed until use.

Generation of Monoclonal Antibodies (mAbs)

The mouse anti-human BTN2 antibodies (clones 4.15, 5.28, 7.28, 7.48, 8.15, 8.16, 8.33 with IgG1 isotype) and mouse anti-human CD277 (also known as BTN3A; clone 20.1 with IgG1 isotype) were purified from cell culture supernatants.

### Flow Cytometry

PBMCs, purified γδ-T cells or tumor cell lines were incubated with specified mAbs before analysis on LSRFortessa™ (Becton Dickinson) using DIVA software (BD bioscience).

Antibodies used for γδ-T cell degranulation assay were: anti-CD107a-FITC (BD Biosciences), anti-CD107b-FITC (BD Biosciences), anti-CD3-PeVio700 (Miltenyi), anti-Tgd-PE (Miltenyi), live/dead near IR (Thermofisher). Antibodies used for screening of BTN2 expression on tumor cell lines were: purified anti-BTN2 (clone 8.16, 10 μg/ml), FcR Block reagent (Miltenyi), goat anti-mouse-PE (Jackson immunoresearch), live/dead near IR (Thermofisher).

### Functional Assay on γδ-T Cells

Purified γδ-T cells from HV were cultured overnight in IL-2 (200 UI/ml). Then, γδ-T cells were co-cultured at 37° C. with Daudi target cell line (at effector:target (E:T) ratio of 1:1), and cytotoxic tests were performed in 4-hours assays in the presence of GolgiStop™ and soluble CD107 (a&b)-FITC, with or without anti-BTN2 (4.15, 5.28, 7.28, 7.48, 8.15, 8.16, 8.33) mAbs and/or anti-CD277 20.1 mAb (10 μg/ml), with or without activation by phosphoagonists (Pag). After 4 hours, cells were collected, fixed and permeabilized then stained with intracellular mAb (IFN-γ, TNF-α). Cells were finally re-suspended in PBS 2% paraformaldehyde and extemporaneously analyzed on a BD LSRFortessa™ (BD Biosciences, San Jose, CA). The degree of cytolytic function of γδ-T cells was measured based on the percentage of cells positive for CD107a and CD107b (degranulation) and/or the production of inflammatory cytokines (IFN-γ, TNF-α).

Binding Test of Anti-BTN2 mAbs on for BTN2A1 and BTN2A2 Expressing Cells

HEK-293F cells bearing CRISPR-Cas9-mediated deletions of all isoforms of BTN3 and BTN2 (293F BTN3/BTN2 KO) were generated (data not shown), cultured in DMEM (Life Technologies) 10% fetal bovine serum (FBS, Gibco) 1 mM sodium pyruvate (Thermofisher scientific), and transfected independently with pcDNA3-Zeo-BTN2A1-CFP or pcDNA3-Zeo-BTN2A2-CFP, which encode BTN2A1 and BTN2A2 CFP(Nter)-fusion proteins, using Lipofectamine™ 2000 reagent (Thermofisher scientific) according to manufacturer's instructions.

Flow Cytometry

Twenty-four hours after transfection, cells (5×10⁴/sample) were collected and stained in duplicate with the indicated concentrations (2-fold dilutions starting from 20 μg/mL to 64 pg/mL) of all 7 purified anti-human BTN2 mAbs in 50 μL of staining buffer (DPBS1× (Thermofisher Scientific) 1% FBS, 1 mM EDTA (Thermofisher Scientific)) during 30 minutes at 4° C. Equal concentrations of mouse IgG1 antibody (Miltenyi) were used as isotype control for staining. Then, cells were washed twice with 200 μL of staining buffer, and incubated with a 1:200 dilution of goat anti-mouse Ig-PE conjugated (Jackson Immunoresearch) in staining buffer for 30 min at 4° C. in the dark. Finally, cells were washed twice in staining buffer prior to fixation using BD Cytofix™ reagent (BD Bioscience) according to manufacturer's instructions. Mean fluorescence intensity (MFI) on PE channel within the CFP-positive population was assessed for each sample in a CytoFLEX LX (Beckman Coulter), and analyzed with a FlowJo™ V10.4.2 software (FlowJo™, LLC 2006-2018).

Statistics

EC₅₀ of purified anti-human BTN2 mAbs on BTN2A1- and BTN2A2-transfected 293F BTN3/BTN2 KO cells were determined based on log(dose) response curves after non-linear regression following a variable-slope model. These analyses were performed using GraphPad Prism 7.04 software (GraphPad).

### Proliferation of γδ-T Cells

γδ-T cells were isolated from PBMCs of healthy donors using anti-TCR γδ microbead kit (Miltenyi Biotec). The purity of γδ-T cells assessed by flow cytometry was greater than 80%. γδ-T cells were labeled with CellTrace™ Violet for 20 minutes at 37° C. Then, 5.10⁵ CellTrace™-labeled cells were cultured in 96-well round-bottom plates in the presence of 200 UI/ml IL-2, with or without Pag, and with or without anti-BTN2 antibodies (10 μg/ml). After 5 days of culture, CellTrace™ dilution was evaluated by flow cytometry on a BD LSRFortessa™ (BD Biosciences, San Jose, CA).

### Statistics

Results are expressed as median±SEM. Statistical analysis was performed using Spearman correlation, Wilcoxon test and Mann-Whitney t test. p values <0.05 were considered significant. Analyses were performed using GraphPad Prism program.

### Results

Identification of the Reference Antibodies mAb1-mAb7

The reference antibodies mAb1-mAb7 were obtained as follows:

Mice were immunized with BTN2A1-Fc antigen. The splenocytes of the mice were collected and fused with myeloma to obtain hybridomas. Hybridomas producing the antibodies with the highest affinity to BTN2 were screened and isolated, yielding the hybridomas as deposited under CNCM I-5231, CNCM I-5232, CNCM I-5233, CNCM I-5234, CNCM I-5235, CNCM I-5236 and CNCM I-5237 capable of producing mAb1-mAb7 respectively.

The hybridoma producing mAb7 (mAb 8.33) which serves as a comparative control for the antibodies according to the present disclosure has been deposited at the Collection Nationale de Cultures des Microorganismes (CNCM, Institut Pasteur, 25 rue du Docteur Roux, 75724 Paris Cedex 15, France) in accordance with the terms of Budapest Treaty on Sep. 14, 2017.

The deposited hybridoma for mAb 8.33 has CNCM deposit number CNCM I-5237.

Binding of mAbs 1-6 on HEK293F Cells.

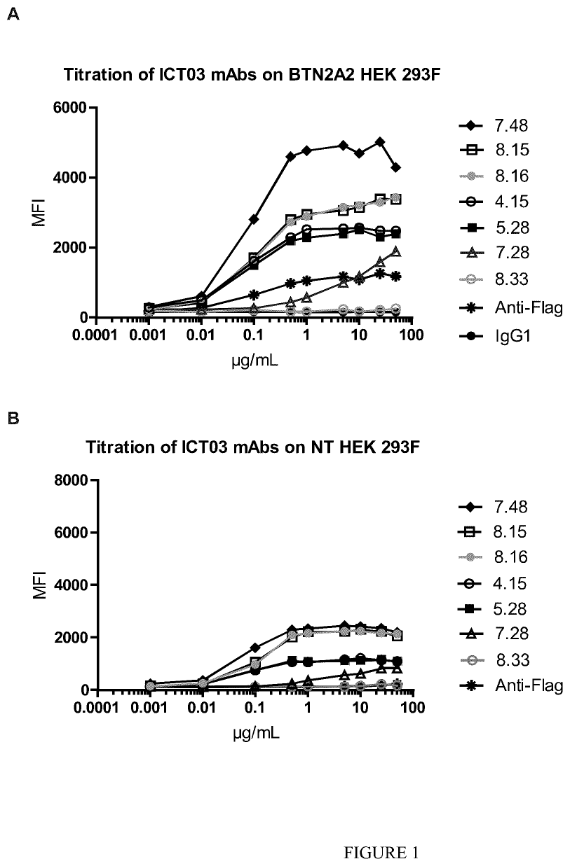

The graphs in FIG. 1 show a titration curve of the affinity of mouse anti-human BTN2 mAbs on HEK293F cells transfected with human BTN2A2 (FIG. 1A) or non-transfected HEK293F cells (FIG. 1B).

The EC₅₀ of each antibody is indicated in the table below. All the antibodies tested were able to recognize and bind to BTN2A2 onto the HEK cells, except mAb 8.33 (mAb7) that does not bind to BTN2A2 by flow cytometry.

| Antibody | Clone Name | EC₅₀ (μg/ml) |
|---|---|---|
| mAb1 | mAb 4.15 | 0.07 |
| mAb2 | mAb 5.28 | 0.07 |
| mAb3 | mAb 7.28 | 31.3 |
| mAb4 | mAb 7.48 | 0.08 |
| mAb5 | mAb 8.15 | 0.1 |
| mAb6 | mAb 8.16 | 0.11 |
| mAb7 | mAb 8.33 | — |

### BTN2 Polypeptide is Expressed on Cancer Cell Lines.

Tumor cell lines were incubated with mAb6 (mouse anti-human BTN2 mAb 8.16) and then with secondary goat anti-mouse-PE. As shown in Figure A, a wide expression of BTN2 protein on panel of tumor cell lines, derivated from solid tumors or hematopoietic malignancies, was observed, including Daudi cell line (Burkitt lymphoma), a standard cell line used in Vγ9Vδ2 T cell degranulation assay.

mAbs 1-6 Inhibit the Cytolytic Function of Vγ9Vδ2 T Cells.

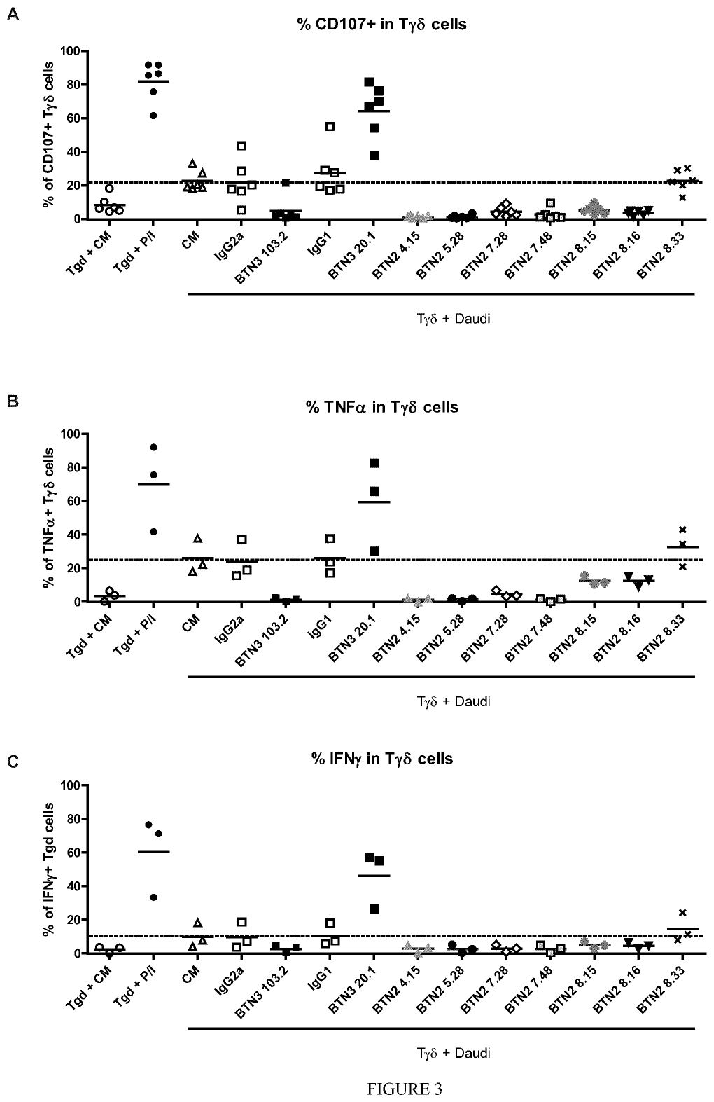

Purified Vγ9Vδ2 T cells were expanded from PBMCs of healthy donors. Vγ9Vδ2 T cells were co-cultured with Daudi target cells. As shown in FIG. 3, the addition of anti-BTN2 mAbs 1-6 lead to an inhibition of the cytolytic function of Vγ9Vδ2 T cells, as measured as CD107 degranulation, and the production of inflammatory cytokines (TNF-α, IFN-γ) against Daudi target cell line.

Control antibody mAb 7 (mAb 8.33), which does not bind to BTN2A2 by flow cytometry, has no effect on Vγ9Vδ2 T cell degranulation (cytolytic function) or production of inflammatory cytokines.

Anti-CD277 20.1 agonist antibody serves as a control example of an activating antibody of Vγ9Vδ2 T cell degranulation, and anti-CD277 103.2 antagonist antibody serves as a control example of an inhibiting antibody of Vγ9Vδ2 T cell degranulation.

mAbs 1-6 Inhibit the Agonist Effect of Anti-CD277 (20.1) mAb on the Cytolytic Function of Vγ9Vδ2 T Cells.

We also tested the effect of the combination of anti-BTN2 mAbs 1-6 in the presence of the agonist anti-CD277 20.1 mAb, (as disclosed in WO2012/080351) and known to increase the degranulation of Vγ9Vδ2 T cells.

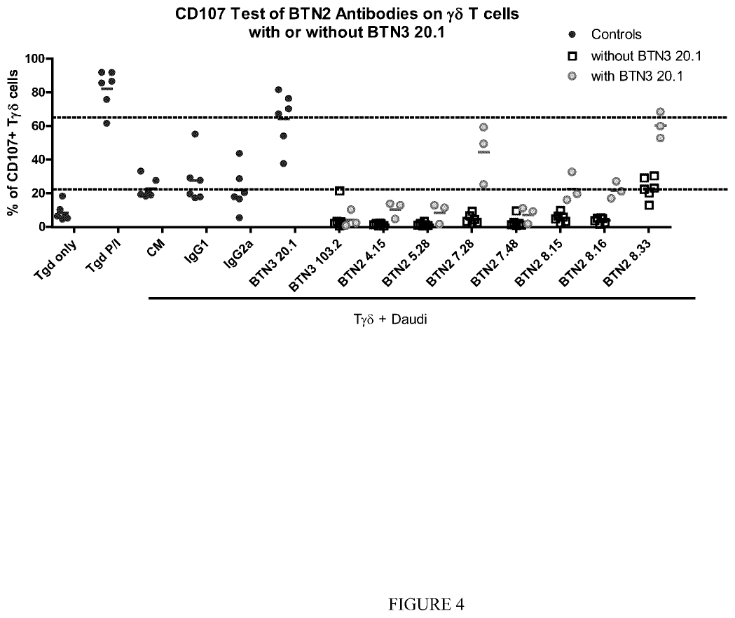

As shown in FIG. 4, mAbs 1, 2 and 4 (respectively anti-BTN2 mAbs 4.15, 5.28, 7.48) surprisingly inhibit the agonist effect of anti-CD277 20.1 antibody on the cytolytic function of Vγ9Vδ2 T cells against Daudi target cell line.

mAbs 5 and 6 (respectively Anti-BTN2 8.15, 8.16) partially inhibit the agonist effect of anti-CD277 20.1 antibody on the cytolytic function of Vγ9Vδ2 T cells against Daudi target cell line.

mAb 3 (anti-BTN2 7.28) and mAb 7 (anti-BTN2 8.33) does not show significant inhibition of the agonist effect of anti-CD277 20.1 antibody on the cytolytic function of Vγ9Vδ2 T cells against Daudi target cell line.

The combination of antagonist 103.2 and agonist 20.1 anti-CD277 serves as a control: antagonist 103.2 antibody inhibits the agonist effect of 20.1 antibody on the cytolytic function of Vγ9Vδ2 T cells against Daudi target cell line, as previously described.

The results of the characterization of mAbs 1-6 (and control mAb 7) are summarized hereafter:

| | | | Effect on |
|---|---|---|---|
| | EC₅₀ (μg/ml) | Effect on | γδ T cells/CD107 |
| Clone | on HEK-BTN2 cells | γδ T cells/CD107 | in presence of 20.1 |
| 4.15 | 0.07 | Inhibition | Inhibition |
| 5.28 | 0.07 | Inhibition | Inhibition |
| 7.28 | 31.30 | Inhibition | No effect |
| 7.48 | 0.08 | Inhibition | Inhibition |
| 8.15 | 0.10 | Inhibition | Partial inhibition |
| 8.16 | 0.11 | Inhibition | Partial inhibition |
| 8.33 | — | No | No effect |

mAbs 1-6 does not Cross-React with HEK293F Cells Transfected with CD277 Isoforms (BTN3A1, BTN3A2 or BTN3A3).

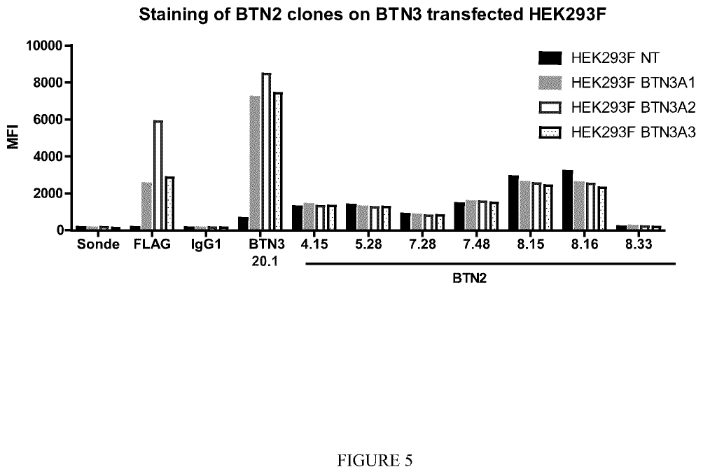

Binding of BTN2 mAbs on HEK293F cells transfected with any one of the isoforms of CD277 (BTN3A1, BTN3A2 or BTN3A3) is similar as the one observed on non-transfected HEK293F cells (see FIG. 5). We can conclude that anti-BTN2 mAbs does not cross-react with one of the isoform of CD277.

mAbs 1-6 has Binding Specificity with Human BTN2A1 and BTN2A2 Isoforms
Binding of mAbs 1-6 on BTN3/BTN2 KO HEK293F Cells.

EC50 of each antibody on BTN2A1 and BTN2A2 are indicated in the table below. All anti-BTN2 antibodies tested were able to bind both isoforms, excepting mAb 8.33 (mAb7), which does not display staining on flow cytometry.

| | | BTN2A1 | BTN2A2 |
|---|---|---|---|
| Antibody | Clone Name | EC₅₀ (μg/ml) | EC₅₀ (μg/ml) |
| mAb1 | mAb 4.15 | 0.06 | 0.01 |
| mAb2 | mAb 5.28 | 0.04 | 0.01 |
| mAb3 | mAb 7.28 | 0.02 | 11.8 |
| mAb4 | mAb 7.48 | 0.02 | 0.01 |
| mAb5 | mAb 8.15 | 0.08 | 0.5 |
| mAb6 | mAb 8.16 | 0.08 | 2.4 |
| mAb7 | mAb 8.33 | — | — |

Summary Table:

| | | | | Tγδ degranulation | |
|---|---|---|---|---|---|
| | | | | (% of inhibition) | |
| | | | | | Against |
| | | | | | target cells |
| | | BTN2A1 | BTN2A2 | Against | (Daudi) + |
| | Clone | EC₅₀ | EC₅₀ | target cells | anti-BTN3 |
| Antibody | Name | (μg/ml) | (μg/ml) | (Daudi) | (20.1) mAb |
| mAb1 | mAb 4.15 | 0.06 | 0.01 | >90% | >90% |
| mAb2 | mAb 5.28 | 0.04 | 0.01 | | |
| mAb3 | mAb 7.28 | 0.02 | 11.8 | | ≤10% |
| mAb4 | mAb 7.48 | 0.02 | 0.01 | | >90% |
| mAb5 | mAb 8.15 | 0.08 | 0.5 | | ≤50% |
| mAb6 | mAb 8.16 | 0.08 | 2.4 | | |
| mAb7 | mAb 8.33 | — | — | No effect | No effect |

### Nucleotides and Amino Acid Sequences for Practicing the Claimed Invention

| SEQ | | |
|---|---|---|
| ID | Brief | |
| NO: | Description | |
| 1 | BTN2A1 aa | MESAAALHFS RPASLLLLLL SLCALVSAQF IVVGPTDPIL |
| | sequence | ATVGENTTLR CHLSPEKNAE DMEVRWFRSQ FSPAVFVYKG |
| | | GRERTEEQME EYRGRTTFVS KDISRGSVAL VIHNITAQEN |
| | | GTYRCYFQEG RSYDEAILHL VVAGLGSKPL |
| | | ISMRGHEDGG IRLECISRGW YPKPLTVWRD PYGGVAPALK |
| | | EVSMPDADGL FMVTTAVIIR DKSVRNMSCS INNTLLGQKK |
| | | ESVIFIPESF MPSVSPCAVA LPIIVVILMI PIAVCIYWIN |
| | | KLQKEKKILS GEKEFERETR EIALKELEKE |
| | | RVQKEEELQV KEKLQEELRW RRTFLHAVDV VLDPDTAHPD |
| | | LFLSEDRRSV RRCPFRHLGE SVPDNPERFD SQPCVLGRES |
| | | FASGKHYWEV EVENVIEWTV GVCRDSVERK GEVLLIPQNG |
| | | FWTLEMHKGQ YRAVSSPDRI LPLKESLCRV |
| | | GVFLDYEAGD VSFYNMRDRS HIYTCPRSAF SVPVRPFFRL |
| | | GCEDSPIFIC PALTGANGVT VPEEGLTLHR VGTHQSL |
| 2 | BTN2A2 aa | MEPAAALHFS LPASLLLLLL LLLLSLCALV SAQFTVVGPA |
| | sequence | NPILAMVGEN TTLRCHLSPE KNAEDMEVRW FRSQFSPAVF |
| | | VYKGGRERTE EQMEEYRGRI TFVSKDINRG SVALVIHNVT |
| | | AQENGIYRCY FQEGRSYDEA ILRLVVAGLG |
| | | SKPLIEIKAQ EDGSIWLECI SGGWYPEPLT VWRDPYGEVV |
| | | PALKEVSIAD ADGLFMVTTA VIIRDKYVRN VSCSVNNTLL |
| | | GQEKETVIFI PESFMPSASP WMVALAVILT ASPWMVSMTV |
| | | ILAVFIIFMA VSICCIKKLQ REKKILSGEK KVEQEEKEIA |
| | | QQLQEELRWR RTFLHAADVV LDPDTAHPEL FLSEDRRSVR |
| | | RGPYRQRVPD NPERFDSQPC VLGWESFASG KHYWEVEVEN |
| | | VMVWTVGVCR HSVERKGEVL LIPQNGFWTL EMFGNQYRAL |
| | | SSPERILPLK ESLCRVGVFL |
| | | DYEAGDVSFY NMRDRSHIYT CPRSAFTVPV RPFFRLGSDD |
| | | SPIFICPALT GASGVMVPEE GLKLHRVGTH QSL |
| 3 | mAb 4.15 | SYDIN |
| | HCDR1 aa | |
| 4 | mAb 4.15 | WIFPGDDSIIQNEKFKG |
| | HCDR2 aa | |
| 5 | mAb 4.15 | LGPLRGFTY |
| | HCDR3 aa | |
| 6 | mAb 4.15 | RASESVDRYGSSFMH |
| | LCDR1 aa | |
| 7 | mAb 4.15 | RASNLES |
| | LCDR2 aa | |
| 8 | mAb 4.15 | QQSNEDPWT |
| | LCDR3 aa | |
| 9 | mAb 4.15 VH | MGWSWVFLFLLSVTAGVHSQVQLQQSGAELVKPGASVKLSCKAS |
| | aa | GYIFTSYDINWVRQRPEQGLEWIGWIFPGDDSIIQNEKFKGKAT |
| | | LITDKSSSTVYMQLSRLTSEDSAVYFCARLGPLRGFTYWGQGTL |
| | | VTVSA |
| 10 | mAb 4.15 VL | METDILLLWVLLLWVPGSTGDIVLIQSPASLAVSLGQRATISCR |
| | aa | ASESVDRYGSSFMHWYQQKPGQPPKLLIYRASNLESGIPARFSG |
| | | SGSRTDFTLTINPVEADDVATYYCQQSNEDPWTFGGGTKLEIK |
| 11 | mAb 5.28 | DYSMN |
| | HCDR1 aa | |
| 12 | mAb 5.28 | RINTETGEPTYADDFKG |
| | HCDR2 aa | |
| 13 | mAb 5.28 | DYAKR |
| | HCDR3 aa | |
| 14 | mAb 5.28 | KASQDVITAVA |
| | LCDR1 aa | |
| 15 | mAb 5.28 | STSYRYT |
| | LCDR2 aa | |
| 16 | mAb 5.28 | LQHYTTPWT |
| | LCDR3 aa | |
| 17 | mAb 5.28 VH | MAWVWTLLFLMAAAQSIQAQIQLVQSGPELKKPGETVKISCKAS |
| | aa | GYTFTDYSMNWVKQAPGKGLKWVGRINTETGEPTYADDFKGRFA |
| | | FSLETSASTAYLQIKNLKNEDTATYFCVRDYAKRWGQGTSVTVS |
| | | S |
| 18 | mAb 5.28 VL | MGIKMESQIQVFVFVSLWLSGVDGDIVMTQSHKFMSTSVGDRVS |
| | aa | ITCKASQDVITAVAWYQQKPGQSPKLLIYSTSYRYTGVPDRFTG |
| | | SGSGTDFTFTISSVQAEDLAVYYCLQHYTTPWTFGGGTKLEIK |
| 19 | mAb 7.28 | SYWIE |
| | HCDR1 aa | |
| 20 | mAb 7.28 | EILPGSGSTKYNEKFRG |
| | HCDR2 aa | |
| 21 | mAb 7.28 | LKGYYGGGAMDY |
| | HCDR3 aa | |
| 22 | mAb 7.28 | RASKSISKYLA |
| | LCDR1 aa | |
| 23 | mAb 7.28 | SGSTLQS |
| | LCDR2 aa | |
| 24 | mAb 7.28 | QQHNEYPWT |
| | LCDR3 aa | |
| 25 | mAb 7.28 VH | MEWTWVFLFLLSVTAGVHSQVHLQQSGAELMKPGASVKISCKAT |
| | aa | GYTFSSYWIEWVKQRPGHGREWIGEILPGSGSTKYNEKFRGKAT |
| | | FAADTSSNTAYVQLSSLTSEDSAVYYCARLKGYYGGGAMDYWGQ |
| | | GTSVTVSS |
| 26 | mAb 7.28 VL | MRFQVQVLGLLLLWISGAQCDVQITQSPSYLAASPGETITINCR |
| | aa | ASKSISKYLAWYQEKPGKTNELLIYSGSTLQSGIPSRFSGSGSG |
| | | TDFTLTISSLEPEDFAMYYCQQHNEYPWTEGGGTKLEIK |
| 27 | mAb 7.48 | DFYMY |
| | HCDR1 aa | |
| 28 | mAb 7.48 | TISDGGSHTYYPDSVKG |
| | HCDR2 aa | |
| 29 | mAb 7.48 | DTTIITPY |
| | HCDR3 aa | |
| 30 | mAb 7.48 | RSSTGAVTTSNYAN |
| | LCDR1 aa | |
| 31 | mAb 7.48 | GTNNRAP |
| | LCDR2 aa | |
| 32 | mAb 7.48 | GLWYSNHWV |
| | LCDR3 aa | |
| 33 | mAb 7.48 VH | MNFGLSLIFLVLVLKGVQCEVQLVESGGDLVKPGGSLKLSCAAS |
| | aa | GFTFSDFYMYWVRRTPEKRLEWVATISDGGSHTYYPDSVKGRFT |
| | | ISRDNAKNNLYLQMRSLKSEDTAMYYCGRDTTIITPYWGQGTLV |
| | | TVSA |
| 34 | mAb 7.48 VL | MAWISLILSLLALSSGAISQSVVTQESALTTSPGETVTLTCRSS |
| | aa | TGAVTTSNYANWVQEKPDHLFTGLIGGTNNRAPGVPARFSGSLI |
| | | GDKAALTITGAQTEDEAIYFCGLWYSNHWVFGGGTKLTVL |
| 35 | mAb 8.15 | GYWMT |
| | HCDR1 aa | |
| 36 | mAb 8.15 | EINPDSSTINYTPSLRD |
| | HCDR2 aa | |
| 37 | mAb 8.15 | GSYYPSY |
| | HCDR3 aa | |
| 38 | mAb 8.15 | RASKSVSSSGYSYMN |
| | LCDR1 aa | |
| 39 | mAb 8.15 | LASNLES |
| | LCDR2 aa | |
| 40 | mAb 8.15 | QHSRELPHT |
| | LCDR3 aa | |
| 41 | mAb 8.15 VH | MDFGLIFFIVALLKGVQCEVKLLESGGGLVQPGGSLKLSCAASG |
| | aa | FDFSGYWMTWVRQAPGKGLEWIGEINPDSSTINYTPSLRDKFII |
| | | SRDNAKNTLYLQMSKVRSEDTALYFCARGSYYPSYWGQGTLVTV |
| | | SA |
| 42 | mAb 8.15 VL | METDTLLLWVLLLWVPGSTGDIVLTQSPASLAVSLGQRATISCR |
| | aa | ASKSVSSSGYSYMNWYQQKPGQPPKLLIYLASNLESGVPARFSG |
| | | SGSGTDFTLNIHPVEDEDAATYYCQHSRELPHTFGGGTKLEIK |
| 43 | mAb 8.16 | GYWMT |
| | HCDR1 aa | |
| 44 | mAb 8.16 | EINPDSSTINYTPSLRD |
| | HCDR2 aa | |
| 45 | mAb 8.16 | GSYYPSY |
| | HCDR3 aa | |
| 46 | mAb 8.16 | RASKSVSSSGYSYMN |
| | LCDR1 aa | |
| 47 | mAb 8.16 | LASNLES |
| | LCDR2 aa | |
| 48 | mAb 8.16 | QHSRELPHT |
| | LCDR3 aa | |
| 49 | mAb 8.33 VH | MDFGLIFFIVALLKGVQCEVKLLESGGGLVQPGGSLKLSCAASG |
| | aa | FDFSGYWMTWVRQAPGKGLEWIGEINPDSSTINYTPSLRDKFII |
| | | SRDNAKNTLYLQMSKVRSEDTALYFCARGSYYPSYWGQGTLVTV |
| | | SA |
| 50 | mAb 8.33 VL | METDTLLLWVLLLWVPGSTGDIVLTQSPASLAVSLGQRATISCR |
| | aa | ASKSVSSSGYSYMNWYQQKPGQPPKLLIYLASNLESGVPARFSG |
| | | SGSGTDFTLNIHPVEDEDAATYYCQHSRELPHTFGGGTKLEIK |
| 51 | mAb 8.33 | SGYYWN |
| | HCDR1 aa | |
| 52 | mAb 8.33 | YISYDGNNNYNPSLKN |
| | HCDR2 aa | |
| 53 | mAb 8.33 | PLYDGYYWYFDV |
| | HCDR3 aa | |
| 54 | mAb 8.33 | ITSTDIDDDMN |
| | LCDR1 aa | |
| 55 | mAb 8.33 | EANTLRP |
| | LCDR2 aa | |
| 56 | mAb 8.33 | LQSDNLPYT |
| | LCDR3 aa | |
| 57 | mAb 8.33 VH | MKVLSLLYLLTAIPGILSDVQLQESGPGLVKPSQSLSLTCSVTG |
| | aa | YSITSGYYWNWIRQFPGNKLEWMGYISYDGNNNYNPSLKNRISI |
| | | TRDTSKNQFFLKLNSVTTEDTATYYCASPLYDGYYWYFDVWGAG |
| | | TTVTVSS |
| 58 | mAb 8.33 VL | MTMFSLALLLSLLLLCVSDSRAETTVTQSPASLSLAIGEKVTIR |
| | aa | CITSTDIDDDMNWYQQKPGEPPKLLISEANTLRPGVPSRFSSSG |
| | | RGTDFVFTIENMLSEDVADYYCLQSDNLPYTFGGGTKLEIK |
| 59 | mAb 4.15 | AGCTATGATATAAAC |
| | HCDR1 nt | |
| 60 | mAb 4.15 | TGGATTTTTCCTGGAGATGATAGTATTATTCAGAATGAGAAGTT |
| | HCDR2 nt | CAAGGGC |
| 61 | mAb 4.15 | TTGGGCCCATTACGAGGGTTTACTTAC |
| | HCDR3 nt | |
| 62 | mAb 4.15 | AGAGCCAGTGAAAGTGTTGATCGTTATGGCAGTAGTTTTATGCA |
| | LCDR1 nt | C |
| 63 | mAb 4.15 | CGTGCATCCAACCTAGAATCT |
| | LCDR2 nt | |
| 64 | mAb 4.15 | CAGCAAAGTAATGAGGATCCGTGGACG |
| | LCDR3 nt | |
| 65 | mAb 4.15 VH | ATGGGATGGAGCTGGGTCTTTCTCTTCCTCCTGTCAGTAACTGC |
| | nt | AGGTGTCCACTCCCAGGTTCAGCTGCAGCAGTCTGGAGCTGAAC |
| | | TGGTAAAGCCTGGGGCTTCAGTGAAGTTGTCCTGCAAGGCTTCT |
| | | GGCTACATCTTCACAAGCTATGATATAAACTGGGTGAGGCAGAG |
| | | GCCTGAACAGGGACTTGAGTGGATTGGATGGATTTTTCCTGGAG |
| | | ATGATAGTATTATTCAGAATGAGAAGTTCAAGGGCAAGGCCACA |
| | | CTGACTACAGACAAATCCTCCAGCACAGTCTACATGCAGCTCAG |
| | | CAGGCTGACATCTGAGGACTCTGCTGTCTATTTCTGTGCAAGAT |
| | | TGGGCCCATTACGAGGGTTTACTTACTGGGGCCAAGGGACTCTG |
| | | GTCACTGTCTCTGCAGGGTTTACTTACTGGGGCCAAGGGACTCT |
| | | GGTCACTGTCTCTGCA |
| 66 | mAb 4.15 VL | ATGGAGACAGACACACTCCTGCTATGGGTGCTGCTGCTCTGGGT |
| | nt | TCCAGGTTCCACAGGTGACATTGTGCTGACCCAATCTCCAGCTT |
| | | CTTTGGCTGTGTCTCTAGGGCAGAGGGCCACCATATCCTGCAGA |
| | | GCCAGTGAAAGTGTTGATCGTTATGGCAGTAGTTTTATGCACTG |
| | | GTACCAGCAGAAACCAGGACAGCCACCCAAACTCCTCATCTATC |
| | | GTGCATCCAACCTAGAATCTGGGATCCCTGCCAGGTTCAGTGGC |
| | | AGTGGGTCTAGGACAGACTTCACCCTCACCATTAATCCTGTGGA |
| | | GGCTGATGATGTTGCAACCTATTACTGTCAGCAAAGTAATGAGG |
| | | ATCCGTGGACGTTCGGTGGAGGCACCAAGCTGGAAATCAAA |
| 67 | mAb 5.28 | GACTATTCAATGAAC |
| | HCDR1 nt | |
| 68 | mAb 5.28 | AGGATCAACACTGAGACTGGTGAGCCAACATATGCAGATGACTT |
| | HCDR2 nt | CAAGGGA |
| 69 | mAb 5.28 | GACTACGCTAAGCGG |
| | HCDR3 nt | |
| 70 | mAb 5.28 | AAGGCCAGTCAGGATGTGATTACTGCTGTAGCC |
| | LCDR1 nt | |
| 71 | mAb 5.28 | TCGACATCCTACCGGTACACT |
| | LCDR2 nt | |
| 72 | mAb 5.28 | CTGCAACATTATACTACTCCGTGGACG |
| | LCDR3 nt | |
| 73 | mAb 5.28 VH | ATGGCTTGGGTGTGGACCTTGCTATTCCTGATGGCAGCTGCCCA |
| | nt | AAGTATCCAAGCACAGATCCAGTTGGTACAGTCTGGACCTGAGC |
| | | TGAAGAAGCCTGGAGAGACAGTCAAGATCTCCTGCAAGGCTTCT |
| | | GGTTATACCTTCACAGACTATTCAATGAACTGGGTGAAACAGGC |
| | | TCCAGGAAAGGGTTTAAAGTGGGTGGGCAGGATCAACACTGAGA |
| | | CTGGTGAGCCAACATATGCAGATGACTTCAAGGGACGGTTTGCC |
| | | TTCTCTTTGGAAACCTCTGCCAGCACTGCCTATTTGCAGATCAA |
| | | AAACCTCAAAAATGAGGACACGGCTACATATTTCTGTGTTAGAG |
| | | ACTACGCTAAGCGGTGGGGTCAAGGAACCTCAGTCACCGTCTCC |
| | | TCA |
| 74 | mAb 5.28 VL | ATGGGCATCAAAATGGAGTCACAGATTCAGGTCTTTGTATTCGT |
| | nt | GTCTCTCTGGTTGTCTGGTGTTGACGGAGACATTGTGATGACCC |
| | | AGTCTCACAAATTCATGTCCACATCAGTAGGAGACAGGGTCAGC |
| | | ATCACCTGCAAGGCCAGTCAGGATGTGATTACTGCTGTAGCCTG |
| | | GTATCAACAGAAACCAGGACAATCTCCTAAACTACTGATTTACT |
| | | CGACATCCTACCGGTACACTGGAGTCCCTGATCGCTTCACTGGC |
| | | AGTGGATCTGGGACGGATTTCACTTTCACCATCAGCAGTGTGCA |
| | | GGCTGAAGACCTGGCAGTTTATTACTGTCTGCAACATTATACTA |
| | | CTCCGTGGACGTTCGGTGGAGGCACCAAGCTGGAAATCAAA |
| 75 | mAb 7.28 | AGCTACTGGATAGAG |
| | HCDR1 nt | |
| 76 | mAb 7.28 | GAGATTTTACCTGGAAGTGGAAGTACTAAGTACAATGAGAAGTT |
| | HCDR2 nt | TAGGGGC |
| 77 | mAb 7.28 | TTGAAGGGTTACTACGGAGGAGGTGCTATGGACTAC |
| | HCDR3 nt | |
| 78 | mAb 7.28 | AGGGCAAGTAAGAGCATTAGCAAATATTTAGCC |
| | LCDR1 nt | |
| 79 | mAb 7.28 | TCTGGATCCACTTTGCAATCT |
| | LCDR2 nt | |
| 80 | mAb 7.28 | CAACAGCATAATGAATACCCGTGGACG |
| | LCDR3 nt | |
| 81 | mAb 7.28 VH | ATGGAATGGACCTGGGTCTTTCTCTTCCTCCTGTCAGTAACTGC |
| | nt | AGGTGTCCACTCCCAGGTTCACCTGCAGCAGTCTGGAGCTGAGC |
| | | TGATGAAGCCTGGGGCCTCAGTGAAAATATCCTGCAAGGCTACT |
| | | GGCTACACATTCAGTAGCTACTGGATAGAGTGGGTAAAGCAGAG |
| | | GCCTGGACATGGCCGTGAGTGGATTGGAGAGATTTTACCTGGAA |
| | | GTGGAAGTACTAAGTACAATGAGAAGTTTAGGGGCAAGGCCACA |
| | | TTCGCTGCAGATACATCCTCCAACACAGCCTACGTGCAACTCAG |
| | | CAGCCTGACATCTGAGGACTCTGCCGTCTATTACTGTGCAAGAT |
| | | TGAAGGGTTACTACGGAGGAGGTGCTATGGACTACTGGGGTCAA |
| | | GGAACCTCAGTTACCGTCTCTTCA |
| 82 | mAb 7.28 VL | ATGAGGTTCCAGGTTCAGGTTCTGGGGCTCCTTCTGCTCTGGAT |
| | nt | ATCAGGTGCCCAGTGTGATGTCCAGATAACCCAGTCTCCATCTT |
| | | ATCTTGCTGCATCTCCTGGAGAAACCATTACTATTAATTGCAGG |
| | | GCAAGTAAGAGCATTAGCAAATATTTAGCCTGGTATCAAGAGAA |
| | | ACCTGGGAAAACTAATGAGCTTCTTATCTACTCTGGATCCACTT |
| | | TGCAATCTGGAATTCCATCAAGGTTCAGTGGCAGTGGATCTGGT |
| | | ACAGATTTCACTCTCACCATCAGTAGCCTGGAGCCTGAAGATTT |
| | | TGCAATGTATTACTGTCAACAGCATAATGAATACCCGTGGACGT |
| | | TCGGTGGAGGCACCAAGCTGGAAATCAAA |
| 83 | mAb 7.48 | GACTTTTACATGTAT |
| | HCDR1 nt | |
| 84 | mAb 7.48 | ACCATTAGTGATGGTGGTAGTCACACCTACTATCCAGACAGTGT |
| | HCDR2 nt | GAAGGGG |
| 85 | mAb 7.48 | GATACTACGATAATTACTCCTTAC |
| | HCDR3 nt | |
| 86 | mAb 7.48 | CGCTCAAGTACTGGGGCTGTTACAACTAGTAACTATGCCAAC |
| | LCDR1 nt | |
| 87 | mAb 7.48 | GGTACCAACAACCGAGCTCCA |
| | LCDR2 nt | |
| 88 | mAb 7.48 | GGTCTTTGGTACAGCAACCATTGGGTG |
| | LCDR3 nt | |
| 89 | mAb 7.48 VH | ATGAACTTCGGGCTCAGCTTGATTTTCCTTGTCCTTGTTTTAAA |
| | nt | AGGTGTCCAGTGTGAAGTGCAGCTGGTGGAGTCTGGGGGAGACT |
| | | TAGTGAAGCCTGGAGGGTCCCTGAAACTCTCCTGTGCAGCCTCT |
| | | GGATTCACTTTCAGTGACTTTTACATGTATTGGGTTCGCCGGAC |
| | | TCCGGAAAAGAGGCTGGAGTGGGTCGCAACCATTAGTGATGGTG |
| | | GTAGTCACACCTACTATCCAGACAGTGTGAAGGGGCGATTCACC |
| | | ATCTCCAGAGACAATGCCAAGAACAACCTCTACCTACAAATGAG |
| | | AAGTCTGAAGTCTGAGGACACAGCCATGTATTACTGTGGAAGAG |
| | | ATACTACGATAATTACTCCTTACTGGGGCCAAGGGACTCTGGTC |
| | | ACTGTCTCTGCA |
| 90 | mAb 7.48 VL | ATGGCCTGGATTTCACTTATACTCTCTCTCCTGGCTCTCAGCTC |
| | nt | AGGGGCCATTTCCCAGTCTGTTGTGACTCAGGAATCTGCACTCA |
| | | CCACATCACCTGGTGAAACAGTCACACTCACTTGTCGCTCAAGT |
| | | ACTGGGGCTGTTACAACTAGTAACTATGCCAACTGGGTCCAAGA |
| | | AAAACCAGATCATTTATTCACTGGTCTAATAGGTGGTACCAACA |
| | | ACCGAGCTCCAGGTGTTCCTGCCAGATTCTCAGGCTCCCTGATT |
| | | GGAGACAAGGCTGCCCTCACCATCACAGGGGCACAGACTGAGGA |
| | | TGAGGCAATATATTTCTGTGGTCTTTGGTACAGCAACCATTGGG |
| | | TGTTCGGTGGAGGAACCAAACTGACTGTCCTA |
| 91 | mAb 8.15 | GGATACTGGATGACT |
| | HCDR1 nt | |
| 92 | mAb 8.15 | GAAATTAATCCAGATAGCAGTACGATAAACTATACGCCATCTCT |
| | HCDR2 nt | AAGGGAT |
| 93 | mAb 8.15 | GGGAGCTACTATCCCTCTTAC |
| | HCDR3 nt | |
| 94 | mAb 8.15 | AGGGCCAGCAAAAGTGTCAGTTCATCTGGCTATAGTTATATGAA |
| | LCDR1 nt | C |
| 95 | mAb 8.15 | CTTGCATCCAACCTAGAATCT |
| | LCDR2 nt | |
| 96 | mAb 8.15 | CAGCACAGTAGGGAGCTTCCGCACACG |
| | LCDR3 nt | |
| 97 | mAb 8.15 VH | ATGGATTTTGGGCTGATTTTTTTTATTGTTGCTCTTTTAAAAGG |
| | nt | GGTCCAGTGTGAAGTGAAGCTTCTCGAGTCTGGAGGTGGCCTGG |
| | | TGCAGCCTGGAGGATCCCTGAAACTCTCCTGTGCAGCCTCAGGA |
| | | TTCGATTTTAGTGGATACTGGATGACTTGGGTCCGGCAGGCTCC |
| | | AGGGAAAGGGCTAGAATGGATTGGAGAAATTAATCCAGATAGCA |
| | | GTACGATAAACTATACGCCATCTCTAAGGGATAAATTCATCATC |
| | | TCCAGAGACAACGCCAAGAATACGCTGTACCTGCAAATGAGCAA |
| | | AGTGAGATCTGAGGACACAGCCCTTTATTTCTGTGCAAGAGGGA |
| | | GCTACTATCCCTCTTACTGGGGCCAAGGGACTCTGGTCACTGTC |
| | | TCTGCA |
| 98 | mAb 8.15 VL | ATGGAGACAGACACACTCCTGTTATGGGTACTGCTGCTCTGGGT |
| | nt | TCCAGGTTCCACTGGGGACATTGTGCTGACACAGTCTCCTGCTT |
| | | CCTTAGCTGTATCTCTGGGGCAGAGGGCCACCATCTCATGCAGG |
| | | GCCAGCAAAAGTGTCAGTTCATCTGGCTATAGTTATATGAACTG |
| | | GTACCAGCAGAAACCAGGACAGCCACCCAAACTCCTCATCTATC |
| | | TTGCATCCAACCTAGAATCTGGGGTCCCTGCCAGGTTCAGTGGC |
| | | AGTGGGTCTGGGACAGACTTCACCCTCAACATCCATCCTGTGGA |
| | | GGATGAGGATGCTGCAACCTATTACTGTCAGCACAGTAGGGAGC |
| | | TTCCGCACACGTTCGGAGGGGGGACCAAGCTGGAAATAAAA |
| 99 | mAb 8.16 | GGATACTGGATGACT |
| | HCDR1 nt | |
| 100 | mAb 8.16 | GAAATTAATCCAGATAGCAGTACGATAAACTATACGCCATCTCT |
| | HCDR2 nt | AAGGGAT |
| 101 | mAb 8.16 | GGGAGCTACTATCCCTCTTAC |
| | HCDR3 nt | |
| 102 | mAb 8.16 | AGGGCCAGCAAAAGTGTCAGTTCATCTGGCTATAGTTATATGAA |
| | LCDR1 nt | C |
| 103 | mAb 8.16 | CTTGCATCCAACCTAGAATCT |
| | LCDR2 nt | |
| 104 | mAb 8.16 | CAGCACAGTAGGGAGCTTCCGCACACG |
| | LCDR3 nt | |
| 105 | mAb 8.33 VH | ATGGATTTTGGGCTGATTTTTTTTATTGTTGCTCTTTTAAAAGG |
| | nt | GGTCCAGTGTGAAGTGAAGCTTCTCGAGTCTGGAGGTGGCCTGG |
| | | TGCAGCCTGGAGGATCCCTGAAACTCTCCTGTGCAGCCTCAGGA |
| | | TTCGATTTTAGTGGATACTGGATGACTTGGGTCCGGCAGGCTCC |
| | | AGGGAAAGGGCTAGAATGGATTGGAGAAATTAATCCAGATAGCA |
| | | GTACGATAAACTATACGCCATCTCTAAGGGATAAATTCATCATC |
| | | TCCAGAGACAACGCCAAGAATACGCTGTACCTGCAAATGAGCAA |
| | | AGTGAGATCTGAGGACACAGCCCTTTATTTCTGTGCAAGAGGGA |
| | | GCTACTATCCCTCTTACTGGGGCCAAGGGACTCTGGTCACTGTC |
| | | TCTGCA |
| 106 | mAb 8.33 VL | ATGGAGACAGACACACTCCTGTTATGGGTACTGCTGCTCTGGGT |
| | nt | TCCAGGTTCCACTGGGGACATTGTGCTGACACAGTCTCCTGCTT |
| | | CCTTAGCTGTATCTCTGGGGCAGAGGGCCACCATCTCATGCAGG |
| | | GCCAGCAAAAGTGTCAGTTCATCTGGCTATAGTTATATGAACTG |
| | | GTACCAGCAGAAACCAGGACAGCCACCCAAACTCCTCATCTATC |
| | | TTGCATCCAACCTAGAATCTGGGGTCCCTGCCAGGTTCAGTGGC |
| | | AGTGGGTCTGGGACAGACTTCACCCTCAACATCCATCCTGTGGA |
| | | GGATGAGGATGCTGCAACCTATTACTGTCAGCACAGTAGGGAGC |
| | | TTCCGCACACGTTCGGAGGGGGGACCAAGCTGGAAATAAAA |
| 107 | mAb 8.33 | AGTGGTTATTACTGGAAC |
| | HCDR1 nt | |
| 108 | mAb 8.33 | TACATAAGCTACGACGGTAACAATAACTACAACCCATCTCTCAA |
| | HCDR2 nt | AAAT |
| 109 | mAb 8.33 | CCTCTCTATGATGGTTATTACTGGTACTTCGATGTC |
| | HCDR3 nt | |
| 110 | mAb 8.33 | ATAACCAGCACTGATATTGATGATGATATGAAC |
| | LCDR1 nt | |
| 111 | mAb 833 | GAAGCCAATACTCTTCGTCCT |
| | LCDR2 nt | |
| 112 | mAb 8.33 | TTGCAAAGTGATAACTTGCCGTACACG |
| | LCDR3 nt | |
| 113 | mAb 8.33 VH | ATGAAAGTGTTGAGTCTGTTGTACCTGTTGACAGCCATTCCTGG |
| | nt | TATCCTGTCTGATGTACAGCTTCAGGAGTCAGGACCTGGCCTCG |
| | | TGAAACCTTCTCAGTCTCTGTCTCTCACCTGCTCTGTCACTGGC |
| | | TACTCCATCACCAGTGGTTATTACTGGAACTGGATCCGGCAGTT |
| | | TCCAGGAAACAAACTGGAATGGATGGGCTACATAAGCTACGACG |
| | | GTAACAATAACTACAACCCATCTCTCAAAAATCGAATCTCCATC |
| | | ACTCGTGACACGTCTAAGAACCAGTTTTTCCTGAAGTTGAATTC |
| | | TGTGACTACTGAGGACACAGCTACATATTACTGTGCAAGTCCTC |
| | | TCTATGATGGTTATTACTGGTACTTCGATGTCTGGGGCGCAGGG |
| | | ACCACGGTCACCGTCTCCTCA |
| 114 | mAb 8.33 VL | ATGACCATGTTCTCACTAGCTCTTCTCCTCAGTCTTCTTCTCCT |
| | nt | CTGTGTCTCTGATTCTAGGGCAGAAACAACTGTGACCCAGTCTC |
| | | CAGCATCCCTGTCCCTGGCTATAGGAGAAAAAGTCACCATCAGA |
| | | TGCATAACCAGCACTGATATTGATGATGATATGAACTGGTACCA |
| | | GCAGAAGCCAGGGGAACCTCCTAAGCTCCTTATTTCAGAAGCCA |
| | | ATACTCTTCGTCCTGGAGTCCCATCCCGATTCTCCAGCAGTGGC |
| | | CGTGGTACAGATTTTGTTTTTACAATTGAAAACATGCTCTCAGA |
| | | AGATGTTGCAGATTACTACTGTTTGCAAAGTGATAACTTGCCGT |
| | | ACACGTTCGGAGGGGGGACCAAGCTGGAAATAAAA |

### REFERENCES

Throughout this application, various references describe the state of the art to which this invention pertains. The disclosures of these references are hereby incorporated by reference into the present disclosure.

1. A method of inhibiting the response of Vγ9/Vδ2 T cells against BTN2-expressing target cells, comprising contacting Vγ9/Vδ2 T cells with BTN2-expressing target cells and an effective amount of an antibody having specificity for human butyrophilin-2 (BTN2), wherein said antibody inhibits the cytolytic function of activated Vγ9/Vδ2 T cells.

2. The method of claim 1, wherein said antibody inhibits the production of IFN-γ and TNF-α of activated Vγ9/Vδ2 T cells and/or inhibits the proliferation of activated Vγ9/Vδ2 T cells.

3. The method of claim 1, wherein said antibody competes for binding to BTN2A2 with at least one of the following reference murine antibodies:
i. mAb 4.15 as obtainable by the hybridoma deposited at the CNCM under deposit number CNCM I-5231;
ii. mAb 5.28 as obtainable by the hybridoma deposited at the CNCM under deposit number CNCM I-5232;
iii. mAb 7.28 as obtainable by the hybridoma deposited at the CNCM under deposit number CNCM I-5233;
iv. mAb 7.48 as obtainable by the hybridoma deposited at the CNCM under deposit number CNCM I-5234;
v. mAb 8.15 as obtainable by the hybridoma deposited at the CNCM under deposit number CNCM I-5235; or
vi. mAb 8.16 as obtainable by the hybridoma deposited at the CNCM under deposit number CNCM I-5236.

4. The method of claim 1, wherein said antibody does not cross-react with human CD277.

5. The method of claim 1, wherein said antibody is a human, chimeric or humanized antibody.

6. A method of inhibiting an immune or inflammatory response in a subject, comprising administering to the subject an effective amount of an antibody having specificity for human butyrophilin-2 (BTN2), wherein said antibody inhibits the cytolytic function of activated Vγ9/Vδ2 T cells.

7. The method of claim 6, wherein said antibody inhibits production of IFN-γ and TNF-α of activated Vγ9/Vδ2 T cells and/or inhibits the proliferation of activated Vγ9/Vδ2 T cells.

8. The method of claim 6, wherein said antibody competes for binding to BTN2A2 with at least one of the following reference murine antibodies:
vii. mAb 4.15 as obtainable by the hybridoma deposited at the CNCM under deposit number CNCM I-5231;
viii. mAb 5.28 as obtainable by the hybridoma deposited at the CNCM under deposit number CNCM I-5232;
ix. mAb 7.28 as obtainable by the hybridoma deposited at the CNCM under deposit number CNCM I-5233;
x. mAb 7.48 as obtainable by the hybridoma deposited at the CNCM under deposit number CNCM I-5234;
xi. mAb 8.15 as obtainable by the hybridoma deposited at the CNCM under deposit number CNCM I-5235; or
xii. mAb 8.16 as obtainable by the hybridoma deposited at the CNCM under deposit number CNCM I-5236.

9. The method of claim 6, wherein said antibody does not cross-react with human CD277.

10. The method of claim 6, wherein said antibody is a human, chimeric or humanized antibody.

11. A method for obtaining an antibody which inhibits the immune response in a subject, wherein said method comprises:
(a) providing one or more antibodies which selectively bind to human butyrophilin-2 (BTN2);
(b) contacting said one or more antibodies with Vγ9/Vδ2 T cells and BTN2-expressing target cells; and
(c) selecting an antibody which inhibits the cytolytic function of activated Vγ9/Vδ2 T cells.

12. The method of claim 11, wherein said selected antibody inhibits production of IFN-γ and TNF-α of activated Vγ9/Vδ2 T cells and/or inhibits the proliferation of activated Vγ9/Vδ2 T cells.

13. The method of claim 1, wherein said selected antibody inhibits the degranulation of activated Vγ9/Vδ2 T cells by more than 90%.

14. The method of claim 11, wherein step (a) comprises immunizing a mouse expressing human variable region antibodies with BTN2 antigens and obtaining a hybridoma producing an antibody which selectively bind to human butyrophilin-2 (BTN2).

15. The method of claim 11, wherein step (a) comprises humanizing an anti-BTN2 antibody.

16. The method of claim 11, wherein step (a) comprises screening antibodies for their binding affinity to BTN2.

17. Wherein the selected antibody competes for binding to BTN2A2 with at least one of the following reference murine antibodies:
xiii. mAb 4.15 as obtainable by the hybridoma deposited at the CNCM under deposit number CNCM I-5231;
xiv. mAb 5.28 as obtainable by the hybridoma deposited at the CNCM under deposit number CNCM I-5232;
xv. mAb 7.28 as obtainable by the hybridoma deposited at the CNCM under deposit number CNCM I-5233;
xvi. mAb 7.48 as obtainable by the hybridoma deposited at the CNCM under deposit number CNCM I-5234;
xvii. mAb 8.15 as obtainable by the hybridoma deposited at the CNCM under deposit number CNCM I-5235; or
xviii. mAb 8.16 as obtainable by the hybridoma deposited at the CNCM under deposit number CNCM I-5236.
